SEQUENCE LISTING (24 sequences)

SEQ ID NO: 1 - DNA, 59 bp, synthetic construct
  source 1..59: mol_type = other DNA
  misc_feature 1..59: note = nucleic acid aptamer
ggtgtgccaataaaccatatcgccgcgttagcatgtactcggttggccctaaatacgag

SEQ ID NO: 2 - DNA, 45 bp, synthetic construct
  source 1..45: mol_type = other DNA
  misc_feature 1..45: note = nucleic acid aptamer
ccggcacgggacaaggcgcgggacggcgtagatcaggtcgacacc

SEQ ID NO: 3 - DNA, 78 bp, synthetic construct
  source 1..78: mol_type = other DNA
  misc_feature 1..78: note = nucleic acid aptamer
gttgctcgtatttagggccaccggcacgggacaaggcgcgggacggcgtagatcaggtcgacaccagtcttcatccgc

SEQ ID NO: 4 - DNA, 76 bp, synthetic construct
  source 1..76: mol_type = other DNA
  misc_feature 1..76: note = nucleic acid aptamer
gcggatgaagactggtgtgccaataaaccatatcgccgcgttagcatgtactcggttggccctaaatacgagcaac

SEQ ID NO: 5 - DNA, 78 bp, synthetic construct; identical to SEQ ID NO: 3
  source 1..78: mol_type = other DNA
  misc_feature 1..78: note = nucleic acid aptamer

SEQ ID NO: 6 - DNA, 78 bp, synthetic construct
  source 1..78: mol_type = other DNA
  misc_feature 1..78: note = nucleic acid aptamer
gcggatgaagactggtgtcgacctgatctacgccgtcccgcgccttgtcccgtgccggtggccctaaatacgagcaac

SEQ ID NO: 7 - DNA, 68 bp, synthetic construct
  source 1..68: mol_type = other DNA
  misc_feature 1..68: note = nucleic acid aptamer
gttgctcgtatttagggcacacacgcacgaagaccttggctgcccgttgtacaccagtcttcatccgc

SEQ ID NO: 8 - DNA, 68 bp, synthetic construct
  source 1..68: mol_type = other DNA
  misc_feature 1..68: note = nucleic acid aptamer
gcggatgaagactggtgtacaacgggcagccaaggtcttcgtgcgtgtgtgccctaaatacgagcaac

SEQ ID NO: 9 - DNA, 76 bp, synthetic construct
  source 1..76: mol_type = other DNA
  misc_feature 1..76: note = nucleic acid aptamer
gttgctcgtatttagggcaccgaggtcaccgaacttggtgtgcacagttgttggcgcgacaccagtcttcatccgc

SEQ ID NO: 10 - DNA, 76 bp, synthetic construct
  source 1..76: mol_type = other DNA
  misc_feature 1..76: note = nucleic acid aptamer
gcggatgaagactggtgtcgcgccaacaactgtgcacaccaagttcggtgacctcggtgccctaaatacgagcaac

SEQ ID NO: 11 - DNA, 76 bp, synthetic construct
  source 1..76: mol_type = other DNA
  misc_feature 1..76: note = nucleic acid aptamer
gttgctcgtatttagggccacatatgtgcacatcacaatccgcagagctgcacctacgacaccagtcttcatccgc

SEQ ID NO: 12 - DNA, 76 bp, synthetic construct
  source 1..76: mol_type = other DNA
  misc_feature 1..76: note = nucleic acid aptamer
gcggatgaagactggtgtcgtaggtgcagctctgcggattgtgatgtgcacatatgtggccctaaatacgagcaac

SEQ ID NO: 13 - DNA, 76 bp, synthetic construct
  source 1..76: mol_type = other DNA
  misc_feature 1..76: note = nucleic acid aptamer
gttgctcgtatttagggccaaggaaaaccccctggtcactggtactaatccgatccgtacaccagtcttcatccgc

SEQ ID NO: 14 - DNA, 76 bp, synthetic construct
  source 1..76: mol_type = other DNA
  misc_feature 1..76: note = nucleic acid aptamer
gcggatgaagactggtgtacggatcggattagtaccagtgaccagggggttttccttggccctaaatacgagcaac

SEQ ID NO: 15 - DNA, 76 bp, synthetic construct
  source 1..76: mol_type = other DNA
  misc_feature 1..76: note = nucleic acid aptamer
gttgctcgtatttagggcgggtcaccacggaagagtgtagatacatagatacagtccgacaccagtcttcatccgc

SEQ ID NO: 16 - DNA, 76 bp, synthetic construct
  misc_feature 1..76: note = nucleic acid aptamer
  source 1..76: mol_type = other DNA
gcggatgaagactggtgtcggactgtatctatgtatctacactcttccgtggtgacccgccctaaatacgagcaac

SEQ ID NO: 17 - DNA, 59 bp, synthetic construct
  source 1..59: mol_type = other DNA
cctgtgccaataaaggataccgccgcgttagcatgtactcggttggccctaaatacgag

SEQ ID NO: 18 - DNA, 59 bp, synthetic construct
  source 1..59: mol_type = other DNA
ggtgtgccaataaaccatatcgccgcgttagcatgtacagggttggccccaaatacctg

SEQ ID NO: 19 - DNA, 60 bp, synthetic construct
  source 1..60: mol_type = other DNA
tcctgtgccaataaaggataccgccgcgttagcatgtactcggttggccctaaatacgag

SEQ ID NO: 20 - DNA, 58 bp, synthetic construct
  source 1..58: mol_type = other DNA
cctgtgccaataaaggataccgccgcgttagcatgtactcggttggcccaaatacgag

SEQ ID NO: 21 - DNA, 61 bp, synthetic construct
  source 1..61: mol_type = other DNA
tcctgtgccaataaaggataccgccgcgttagcatgtactcggttggccctaaatacgagt

SEQ ID NO: 22 - DNA, 59 bp, synthetic construct
  source 1..59: mol_type = other DNA
ggtgtgccaataaaccataccgccgcgttagcatgtacctggttggccccaaatacagg

SEQ ID NO: 23 - DNA, 76 bp, synthetic construct
  source 1..76: mol_type = other DNA
tttttcttttttcggtgtgccaataaaccatgctaccgcgttagcatgtactcggttggcctcaaatacgagtttt

SEQ ID NO: 24 - DNA, 76 bp, synthetic construct
  source 1..76: mol_type = other DNA
ataagcaggagtcggtgtgccaataaaccatgctaccgcgttagcatgtactcggttggcctcaaatacgagtggt

(19) United States
(12) Patent Application Publication
(10) Pub. No.: US 2026/0218202 A1
(43) Pub. Date: Jul. 30, 2026

(54) APTOLL MOLECULES FOR THE TREATMENT OF ISCHEMIC STROKE AND INTRACRANIAL HEMORRHAGES

(71) Applicant: MERCK PATENT GMBH, Darmstadt (DE)

(72) Inventors: Macarena Hernández Jiménez, Madrid (ES); David Segarra De La Peña, Madrid (ES); María Eugenia Zarabozo Leal, Madrid (ES)

(21) Appl. No.: 19/145,792

(22) PCT Filed: Jan. 4, 2024
(86) PCT No.: PCT/EP2024/050174

§ 371 Date: Jul. 3, 2025

(30) Foreign Application Priority Data
Jan. 5, 2023 (EP) 23382006.7

(51) Int. Cl. C12N 15/115; A61K 38/48; A61P 9/10

(52) U.S. Cl. CPC C12N 15/115; A61K 38/482; A61P 9/10

(57) ABSTRACT

The APRIL study showed that the infusion of ApTOLL 0.2 mg/kg (SEQ ID NO: 1) in combination with endovascular treatment in selected stroke patients is safe and reduces the mortality at 90 days. ApTOLL has demonstrated that limit the phenomena of intracranial hemorrhages and reperfusion damage that may occur after recanalization, and also improves early neurological impairment and long term disability. ApTOLL molecules are provided for use in reducing the risk of intracranial or secondary hemorrhages, in reducing the neurological damage and in improving the neurological recovery after an acute ischemic stroke in a subject.

### FIELD OF THE INVENTION

The present invention relates to the fields of oligonucleotides and therapeutic aptamers for human health, particularly useful in the treatment of intracranial hemorrhages and the improvement of the neurological recovery in patients having suffered a thrombotic disorder, particularly an acute ischemic stroke.

### BACKGROUND ART

Reperfusion treatments have substantially improved acute ischemic stroke (AIS) care over the last two decades. Since 1995 tissue plasminogen activator (tPA) has been the only approved specific treatment for AIS, which has positively impacted the lives of many stroke patients. However, its benefit has been severely curtailed by its limited therapeutic time-window set at <4.5 h after stroke onset and its limited efficacy. It is estimated that tPA is only used in about 5% of AIS patients.

It was not until 2015 when most clinical guidelines recommended also mechanical thrombectomy as a first-line treatment in ischemic strokes due to a large vessel occlusion (LVO). In recent years, the number of patients that benefit from endovascular thrombectomy (EVT) is rapidly growing worldwide and its indications are progressively being expanded to patients presenting with a large infarct core or presenting in a late time window up to 24 hours from symptom onset.

However, these treatments are by no means entirely free of complications. The most significant concern about the use of recanalization therapies such as tPA or EVT is the increased risk of hemorrhage, commonly known as hemorrhagic transformation (HT), which represents the conversion of a stroke into an area of hemorrhage. Other hemorrhages associated to ischemic stroke can also appear; this is the case of intracranial hemorrhages (ICH), that refers to any bleeding within the intracranial vault, including the brain parenchyma and surrounding meningeal spaces. Therefore, both types of hemorrhages (hereinafter referred to as ICH in the present document) occur after an ischemic stroke, which can also be named secondary hemorrhages, and can result in new symptoms or in worsening of the existing ones. Patients that suffer secondary hemorrhages can result in greater neurological damage and more difficult neurological recovery, even death. Several trials concluded that ICH was the presumed responsible cause of neurological deterioration (≥4 points in NIHSS score).

Further, although proven effective, tPA has a limited efficacy in recanalizing LVO. In fact, the larger is the occluding clot, meaning a more proximal occlusion, the lower is the rate of early recanalization after thrombolysis. Several studies have described recanalization rates as low as 10% for terminal internal carotid artery (TICA) occlusion or 30% for M1-MCA (middle cerebral artery M1 segment) occlusion in the hours following intravenous (i.v.) tPA treatment.

Endovascular reperfusion therapies are currently considered fully implemented meaning that acute stroke patients are now consistently undergoing a complete vascular evaluation (computed tomography angiography and CT perfusion [CTA/CTP]) and those presenting LVO undergo EVT that ensures recanalization rates as high as 85-90%. Moreover, several studies show the greater therapeutic window of EVT (up to 24 h after stroke onset in some specific cases) compared with 4.5 h in the case of tPA. However, even though EVT has shown to consistently achieve substantial recanalization grades in 85-90% of the cases, more than 50% of the treated patients will develop a moderate to severe disability.

Despite this known inherent risk of reperfusion therapies, the benefit-risk ratio favors their use. In this context, it is of great interest to find new drugs with potential neuroprotective effects to improve stroke outcomes while reducing intracranial hemorrhages, in parallel with reperfusion treatments.

ApTOLL, an anti-inflammatory drug with proven neuroprotective effects in preclinical models, is an aptamer targeting the extracellular domain of TLR-4, a receptor involved in innate immune responses that also responds to tissue damage-associated molecular patterns (DAMPs) and which is directly involved in a large number of diseases such as ischemic stroke. Specifically, the inflammatory component triggered in the acute phase of stroke is considered an interesting target to enhance the recovery of patients, and ApTOLL offers a potential high therapeutic effect in this area. WO2015197706A1 (AptaTargets SL) describes nucleic acid aptamers, particularly ApTOLL, with the capability of binding specifically to and inhibiting TLR-4 and uses thereof, including stroke.

In fact, the efficacy of ApTOLL has been demonstrated at the preclinical level in experimental models of cerebral and myocardial ischemia. Particularly, WO2020230108A1 (AptaTargets SL) discloses methods and compositions for the treatment of ischemic stroke and demonstrates the efficacy of ApTOLL in rodent models of stroke (Example 2). In addition, a First-in-Human study showed the safety and defined the pharmacokinetics of ApTOLL in healthy subjects, described in Example 3.1 of WO2020230108A1 and in Hernández-Jiménez M, et al., 2022.

### SUMMARY OF THE INVENTION

One problem to be solved by the present invention is to provide new uses and methods that improve functional outcomes of patients, e.g., reduction of neurological damage, while reducing hemorrhagic complications associated to an ischemic stroke and to reperfusion techniques.

The solution is based on the administration of ApTOLL molecules (e.g., ApTOLL, SEQ ID NO: 1 at 0.2 mg/kg) to a subject in need thereof for reducing neurological damage and intracranial hemorrhages among other effects explained hereinafter, associated to an ischemic stroke and to reperfusion techniques.

In this scenario, inventors have designed a Phase Ib/IIa clinical study coded APRIL, hereinafter APRIL study, to assess the safety of ApTOLL and its biological effects in AIS patients who are eligible for EVT with or without i.v. thrombolysis. The protocol for this clinical study was described in Example 3.2 of WO2020230108A1 (AptaTargets SL), but the effects remain unknown.

APRIL study was divided in two parts:
(1) Phase Ib (n=32 AIS patients): a single i.v. administration of ApTOLL (30 min infusion), dose escalation with 4 single dose levels (8 patients/level), randomized (1:3); and
(2) Phase IIa (n=119 patients): a single dose, i.v. administration (30 min infusion), parallel (3 arms, placebo: ApTOLL dose A: ApTOLL dose B), randomized (√2:1:1).

After completion of phase Ib, a data safety monitoring board (DSMB) unblinded to study groups selected 2 doses (A, B) to be tested in phase IIa according to initial safety results. Patients treated with placebo, dose A or dose B in the Phase Ib were further analyzed together with patients enrolled in the Phase IIa (total number of patients: 151).

The primary objective of APRIL study was to evaluate if administration of ApTOLL i.v. at different doses was safe and well tolerated compared to placebo when administered with endovascular therapy, and i.v. fibrinolysis when indicated. APRIL study was not powered to achieve conclusions regarding the efficacy of ApTOLL in improving outcomes in acute stroke patients.

Surprisingly, beyond demonstrating safety of ApTOLL, inventors found that 0.2 mg/kg of ApTOLL administered within 6 h of onset, in combination with EVT, was associated with meaningful clinical effects reducing mortality and disability at 90 days as compared to placebo. ApTOLL is able to increase survival, reduce intracranial hemorrhages, reduce brain edema, and improve the neurological recovery, among other effects, in human patients having suffered AIS. Working examples herein provide the results of APRIL study with detailed experimental data demonstrating said effects of ApTOLL (e.g., EXAMPLE 1, sections 1.13-1.15).

Particularly, inventors have found that ApTOLL reduces intracranial hemorrhages, and remarkably reduces symptomatic intracranial hemorrhages in a rate of 34.25% compared to placebo.

Further, they have found that ApTOLL reduces the number of deaths: death of any cause occurred in 10 patients allocated to placebo (18.2%) and in 2 patients allocated to ApTOLL 0.2 mg/kg (4.8%; absolute difference vs. placebo −13%; 95% CI: −25% to −1%), representing a reduction in deaths of 73%.

The final infarct volume was 44 mL in patients allocated to placebo, and 23.5 mL in patients allocated to ApTOLL 0.2 mg/kg (mean difference of log-transformed final infarct volume vs. placebo −42%; 95% CI: −66% to 1%), resulting in a reduction in final infarct volume of 46.59%.

The NIHSS score (National Institute of Health Stroke Scale) assessed at 72 hours was 7 in patients allocated to placebo and 3 in patients allocated to ApTOLL 0.2 mg/kg (mean difference of log-transformed 72 hours NIHSS vs. placebo −45%; 95% CI: −67% to −10%). This represents a reduction in NIHSS score of 57.14% compared to placebo.

The rate of patients with mRs score (modified Rankin Score) 0-2 (no symptoms-slight disability) at 90 days was 47.1% in patients allocated to placebo and 64.3% in patients allocated to ApTOLL 0.2 mg/kg. This represents an increase (i.e., improvement) in the mRS score of 0-2 of 36.29% compared to placebo.

Further, ApTOLL 0.2 mg/kg caused a reduction in brain edema of 67.40% compared to placebo.

The subgroup analysis suggests a similar treatment effect in patients who received ApTOLL in the early time window (<3 hours from symptom onset) and those who were treated in the later window (3-6 hours), which suggests that ApTOLL is applicable to different clinical situations.

These observed effects of ApTOLL are surprising, considering the seriousness of the disease studied in the clinical trial, where patients suffer from AIS, a very severe and acute disease, and also considering the limited number of participants (151). These effects are clinically meaningful for AIS patients and of significant impact since represents an increase in survival and a reduction in functional impairment and disability, which represents an unmet medical need. It also represents an improvement on the widely used reperfusion techniques such as EVT and thrombolysis. This is crucial because these patients currently have limited treatment options, and our findings highlight the potential significance of ApTOLL in addressing this unmet medical need, i.e., a long-felt need.

As discussed above, WO2020230108A1 demonstrates the efficacy of ApTOLL in rodent models of stroke (Example 2). ApTOLL induced the reduction of infarct size vs. vehicle, revealing its potential protective effect. Moreover, mice preserved the protective effect up to 21 days after stroke. WO2020230108A1 together with Hernández-Jiménez M, et al., 2022 also describe the results of a First-in-Human study of ApTOLL. This study was aimed at defining safety and pharmacokinetics of ApTOLL in healthy subjects.

Thus, results from the APRIL study are the first results directed to the efficacy of ApTOLL in human subjects having suffered AIS. The observed effects are new with respect to the prior art and the skilled person could not have foreseen the herein experimentally described effects from the prior art. In addition to the fact that human subjects have suffered AIS, whose biochemical/clinical situation is different from healthy subjects, the conditions, requirements, doses and inclusion criteria in the APRIL study also differs from those described in the prior art; e.g., EVT and fibrinolysis techniques are performed in APRIL study which can compromise further the patient causing e.g., intracranial hemorrhages. Further, most of the observed effects are associated with the ApTOLL dose of 0.2 mg/kg.

Therefore, the present invention is directed to new effects that were completely unforeseen, particularly the reduction of the risk of intracranial hemorrhages. The causes of these effects triggered by ApTOLL are unknown, suggesting that ApTOLL may have the ability to interact with other receptors besides TLR-4 to e.g., prevent the development of intracranial hemorrhages after an AIS.

The improvement in the neurological recovery was also a remarkable result that is neither derivable from the prior art, since the effect was not necessarily present in previous studies with ApTOLL given the scenario of the clinical trial. For instance, working examples herein specify the reduction in infarct volume, NIHSS score and mRS score, values that suggest a significant neurological improvement in AIS patents. Moreover, results also suggest that the improvement is not only with respect to mobility, but also cognitive.

Thus, the observed effects in APRIL study, i.e., the reduction in the ICH and the reduction in neurological damage and improvement in the neurological recovery among others, can be considered as different technical effects not anticipated. A different technical effect can represent a further medical indication or a new use of a known substance in the sense e.g., of EPO Guidelines.

Working EXAMPLE 2 compares the effects of nerinetide and ApTOLL from ESCAPE-NA1 and APRIL studies, respectively. ESCAPE-NA1 was a phase III clinical trial with 1105 patients but with very limited results since effects were observed only in patients not treated with tPA. Therefore, the applicability of nerinetide to clinical practice in the future would be limited to said kind of patients. The results in patients not treated with tPA were a 19% increase in 0-2 mRS and a 37% reduction of mortality. Contrarily, APRIL study conducted with only 151 patients resulted in a 36% increase in 0-2 mRS (i.e., the mRS score was reduced to 0-2 mRS) and a 72% reduction in mortality over patients either treated or not treated with tPA. EXAMPLE 2 remarks that the effects of APRIL study are positive and applicable to a broader range of patients and medical scenarios.

As discussed above, although APRIL protocol is described in Example 3.2 of WO2020230108A1, outcomes associated to the defined objectives and endpoints are not anticipated. Of note, said outcomes have been unexpected and of significant impact for AIS patients, to the point that the results have exceeded any possible expectation of the skilled clinicians participating in the study.

These surprising effects are neither derivable from Garcia-Culebras A et al., 2017. This document describes that late tPA administration in TLR-mice did not result in worse hemorrhages as occurred in TLR-4^{+/+} group. However, the study presents some limitations, also cited in the document:
(1) the delayed administration was set at the time still safe in humans;
(2) the protective effect of TLR-4 absence requires to be studied in future long-term outcome experiments; and
(3) TLR-4 involvement in thrombectomy-induced HT remains to be studied.

In brief, this study was carried out in mouse models that have a total knock-out of TLR-4 receptor and therefore the mechanisms are not the same as in a normal scenario, e.g., these mice do not undergo an inflammatory response in the TLR-4 pathway when a stroke is triggered. Instead, the APRIL study uses ApTOLL in AIS patients: thus, in the first place, the aptamer partially inhibits TLR-4 and thus does not have the same effects as a complete absence of TLR-4 in all tissues; and secondly, patients experience inflammation in the TLR-4 pathway in brain and have multiple complications in clinical practice, contrary to the mouse model of García-Culebras A et al., 2017.

Accordingly, a first aspect of the invention relates to ApTOLL molecules for use in reducing the risk of intracranial or secondary hemorrhages (or preventing intracranial hemorrhages or reducing the occurrence of intracranial hemorrhages) after an acute ischemic stroke in a subject, wherein the ApTOLL molecules are administered after the acute ischemic stroke (i.e., after stroke onset).

Another aspect relates to ApTOLL molecules for use in reducing the risk of intracranial or secondary hemorrhages (or preventing intracranial hemorrhages or reducing the occurrence of intracranial hemorrhages) after a thrombotic disorder in a subject, wherein the ApTOLL molecules are administered after the thrombotic disorder.

An aspect of the invention is related to ApTOLL molecules for use in reducing the neurological damage and in improving the neurological recovery an after acute ischemic stroke in a subject, wherein the ApTOLL molecules are administered after the acute ischemic stroke.

In another aspect, the present invention relates to ApTOLL molecules for use in increasing survival and/or reducing brain edema after an acute ischemic stroke in a subject, wherein the ApTOLL molecules are administered after the acute ischemic stroke.

Other aspects of the invention relate to ApTOLL molecules for use in improving anxiety/depression complications, performance of usual activities, mobility and/or self care after an acute ischemic stroke in a subject, wherein the ApTOLL molecules are administered after the acute ischemic stroke.

Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. Furthermore, the present invention covers all possible combinations of particular and preferred embodiments described herein. The following examples and drawings are provided herein for illustrative purposes, and without intending to be limiting to the present invention.

### DESCRIPTION OF DRAWINGS

FIG. 1 shows the APRIL study flowchart (Pbo: Placebo, DSMB: Data Safety Monitoring Board, AIS; Acute ischemic stroke).

FIG. 2 represents the median and interquartile range of baseline and 72 hours NIHSS score according to treatment allocation and the median and interquartile range of baseline predicted infarct core on CT-Perfusion and final infarct volume (magnetic resonance imaging at 72 hours) according to treatment allocation. NIHSS: National Institute of Health Stroke Scale.

FIG. 3 shows the distribution of global disability at 90 days according to treatment allocation. Stacked bar plots represent the distribution of the modified Rankin Score (scale from 0 to 6) at 90 days according to treatment allocation.

FIG. 4 is a graphical representation of the quality of life evaluation of patients after treatment with ApTOLL 0.05 mg/kg, 0.02 mg/kg or Placebo. Quality of life evaluation includes the following criteria: mobility, self care, usual activities, pain/discomfort, and anxiety/depression.

FIG. 5 shows detailed flowcharts of the procedure in APRIL study. LVO: large vessel occlusion. TICA: terminal internal carotid artery. mRS: modified Rankin Score. NIHSS: National Institute of Health Stroke Scale. CT/CTA/CTP: computerized tomography/computerized tomography angiography/computed tomography perfusion. MRI: magnetic resonance imaging. ASPECTS: Alberta stroke program early CT score. CBF: cerebral blood flow. DWI: diffusion-weighted imaging. rt-PA (recombinant tissue plasminogen activator. EVT: endovascular thrombectomy.

FIG. 6 shows the primary, secondary and tertiary structure of ApTOLL (SEQ ID NO: 1), also referenced as ApTLR #4 FT.

FIG. 7 shows a comparison in mRS and survival outcomes after administration of 0.2 mg/kg ApTOLL in the APRIL study vs 2.6 mg/kg Nerinetide (NA-1) administration in ESCAPE-NA1 study. mRS: modified Rankin Score. tPA: tissue plasminogen activator. EVT: endovascular thrombectomy.

FIG. 8 represents the antagonist TLR4 activity assay of ApTOLL (SEQ ID NO: 1) and its variants ApTOLL-Mut 1-6 (SEQ ID NO: 17-22). The activity of TLR4 receptor is represented in percentage with respect to control LPS-Ek up. The antagonist activity of the aptamer is identified by the decrease of activation percentage in regard to LPS-EK up.

FIG. 9 represents the competition assay for TLR4 receptor of ApTOLL (SEQ ID NO: 1) and its variants ApTOLL-Mut 1-6 (SEQ ID NO: 17-22). The mutants competition with ApTOLL sequence for the same binding site in TLR4 receptor is identified by decrease of binding percentage of ApTOLL sequence (control) in each mix ApTOLL/ApTOLL-Mut (1-6) in respect to ApTOLL.

### DETAILED DESCRIPTION OF THE INVENTION

The present invention is directed to uses and methods of ApTOLL molecules (e.g., SEQ ID NO: 1) in reducing the risk of intracranial hemorrhages after AIS in a subject; uses and methods of ApTOLL molecules in reducing the risk of intracranial hemorrhages after a thrombotic disorder in a subject; uses and methods of ApTOLL molecules in reducing the neurological damage and in improving the neurological recovery after AIS in a subject (e.g., decrease in infarct volume and NIHSS score and improvement in mRS score); and uses and methods of ApTOLL molecules in increasing survival, and/or reducing brain edema, after AIS in a subject, wherein the ApTOLL molecules are administered after AIS (or after a thrombotic disorder). Also provided are administration procedures and doses of ApTOLL molecules (e.g., 0.2 mg/kg); artery recanalization techniques (e.g., EVT or thrombolysis); subject characterization (e.g., pre-stroke mRS score); and nucleic acid aptamers, variants, derivatives, chemically modified aptamers, pharmaceutical compositions and formulations thereof.

Before the present invention is described in greater detail, it is to be understood that this disclosure is not limited to the particular compositions or process steps described, as such can, of course, vary. As will be apparent to those of skill in the art upon reading this description, each of the individual aspects described and illustrated herein has discrete components and features which can be readily separated from or combined with the features of any of the other several aspects without departing from the scope or spirit of the present description. Any recited method can be carried out in the order of events recited or in any other order which is logically possible.

The headings provided herein are not limitations of the various aspects of the description, which can be defined by reference to the specification as a whole. It is also to be understood that the terminology used herein is for the purpose of describing particular embodiments only, and is not intended to be limiting, since the scope of the present description will be limited only by the appended claims.

Accordingly, the terms defined immediately below are more fully defined by reference to the description in its entirety.

### Definitions

In order that the present description can be more readily understood, certain terms are first defined. Additional definitions are set forth throughout the detailed description.

Treatment: The terms “treat”, “treatment”, “therapy”, as used herein refers to a clinical intervention to prevent (e.g., suppress or inhibit) a disease or condition (e.g., intracranial hemorrhages); cure the disease or condition; delay the onset of the disease or condition; reduce the seriousness or severity of the disease or condition (e.g., reduce the extent of intracranial hemorrhages); ameliorate or eliminate one or more symptoms or sequelae associated with a disease or condition; or the provision of beneficial effects to a subject with a disease or condition, without necessarily curing the disease or condition.

In some embodiments, the term refers to a clinical intervention to improve one or more symptoms; improve one or more sequelae; prevent (e.g., suppress, inhibit or delay) one or more symptoms; prevent (e.g., suppress, inhibit or delay) one or more sequelae; delay one or more symptoms; delay one or more sequelae; ameliorate one or more symptoms; ameliorate one or more sequelae; shorten the duration one or more symptoms; shorten the duration of one or more sequelae; reduce the frequency of one or more symptoms; reduce the frequency of one or more sequelae; reduce the severity of one or more symptoms; reduce the severity of one or more sequelae; improve the quality of life; increase survival; prevent (e.g., suppress, inhibit or delay) a recurrence of the disease or condition; delay a recurrence of the disease or condition; reduce the severity of the disease (e.g., reduce the extent of intracranial hemorrhages); or any combination thereof, e.g., with respect to what is expected in the absence of the treatment with ApTOLL molecules of the present invention.

The term “treatment” also includes prophylaxis or prevention (e.g., suppression, inhibition or delay) of a disease or condition or its symptoms or sequelae thereof. Prophylaxis refers to a therapeutic or course of action used to prevent, inhibit, suppress, reduce the risk, reduce the occurrence or delay the onset of a disease or condition, e.g., intracranial hemorrhages, or to prevent, inhibit, suppress, or delay a symptom associated with a disease or condition.

In some embodiments, the disease or condition are intracranial hemorrhages after a thrombotic disorder, particularly, an ischemic stroke. In some embodiments, ApTOLL molecules are for use in the treatment of intracranial hemorrhages, or in preventing intracranial hemorrhages, or in reducing the occurrence of intracranial hemorrhages, or in reducing the extent of intracranial hemorrhages after a thrombotic disorder, e.g., an ischemic stroke.

In other embodiments, the disease or condition is neurological damage after an acute ischemic stroke. In some embodiments, ApTOLL molecules are for use in the treatment of neurological damage, or in reducing the neurological damage, or in improving the neurological recovery after an ischemic stroke.

Ischemic stroke: this term as used herein refers to a type of stroke (also known as cerebrovascular disease, cerebral infarction, cerebral attack, or apoplexy) characterized by a neurological deficit caused by an important decrease in cerebral blood flow in an abnormally abrupt manner. In ischemic stroke, blood irrigation is lost due to the sudden and immediate interruption of blood flow due to occlusion of any of the arteries irrigating the brain mass, which generates the appearance of an infarcted area. Artery occlusion is generally due to atherosclerosis or an embolus (cerebral embolism) that originates in another location, generally the heart or other arteries. Ischemic stroke is a pathology characterized by an increase in the expression of TLR-4 and/or increase in activation of TLR-4. Given that activation of TLR-4 produces a signaling cascade resulting in the release of inflammatory cytokines such as IL-1, IL-8, TNF-alpha, IL-6, and IL-12, and in the activation and/or recruitment of inflammation cells, causing inflammation and cell damage, the pathology characterized by an increase in expression of TLR-4 and/or an increase in activation of TLR-4 can furthermore be characterized by having an inflammatory component. “Acute ischemic stroke” refers to the first phase of an ischemic stroke, where the treatment (e.g., with ApTOLL molecules) is given to the patient. However, “Ischemic stroke” and “Acute ischemic stroke” are used interchangeable herein. Acute ischemic stroke will be hereinafter referred to as AIS.

Stroke onset: this term as used herein refers to the time at which the stroke is triggered. In clinical practice, stroke onset corresponds to the symptom onset, that is the first clinical symptom or sign for a particular condition, in this case stroke, i.e., time of symptoms onset or last time seen well (LTSW). Since the variation between stroke onset and symptoms onset is minimum, the terms “stroke onset” and “symptom onset” are used interchangeably herein. Some stroke symptoms include sudden numbness or weakness in the face, arm, or leg, especially on one side of the body, sudden confusion, trouble speaking, or difficulty understanding speech, sudden trouble seeing in one or both eyes, sudden trouble walking, dizziness, loss of balance, or lack of coordination, sudden severe headache with no known cause, among other symptoms.

In APRIL study, some parameters are defined with respect to symptoms onset, such as EVT, pharmacological thrombolysis and inclusion criteria.

In APRIL study, some timepoints are defined with respect to the allocation of the patient to the trial (similar to baseline) instead of stroke onset. These are the final infarct volume (measured at 72 h from allocation), the NIHSS score (measured at 72 h from allocation) and the mRS score (measured at 90 d from allocation).

In some cases, allocation of the patient is close to symptoms onset, e.g., inclusion criteria of infarct volume between 5 cc and 70 cc is measured before allocation, which is close to symptoms onset. Therefore, the terms “stroke onset”, “symptoms onset”, “allocation” and “baseline” can be used interchangeably in these cases.

Intracranial hemorrhages (ICH): this term as used herein refers to hemorrhages that occur intracerebrally spontaneously due to complications of ischemic stroke and often triggered by reperfusion therapies such as thrombolysis or EVT. ICH refers to any bleeding within the intracranial vault, including the brain parenchyma and surrounding meningeal spaces. Since ICH in this description are due to an ischemic stroke and they are not the primary cause of disease, they are also referred in this description as “secondary hemorrhages”. ICH can be symptomatic ICH (sICH) or asymptomatic ICH (aICH). ICH can result in new symptoms or in worsening of the existing ones, which are then called sICH. Truly ICH is associated with distinct deterioration in neurological status as established by the investigator in terms of a worsening of the NIHSS by ≥4 points.

In this description, ICH also include hemorrhagic transformation (HT). HT represents the conversion of a stroke into an area of hemorrhage. HT is a common complication of ischemic stroke that is often exacerbated by reperfusion with thrombolysis or EVT. It occurs when the blood-brain barrier (BBB) is sufficiently disrupted to permit extravasation of peripheral blood into the brain. When HT occurs, it increases stroke morbidity and mortality.

In some embodiments, intracranial hemorrhages are symptomatic. In some embodiments intracranial hemorrhages are hemorrhagic transformation.

Intracranial hemorrhages can be measured by imaging procedures, such as non-contrast CT, CTA, CPT and MRI. Intracranial hemorrhages can be classified e.g., according to the Heidelberg Bleeding Classification.

Time window: this term as used herein, also referred to as “therapeutic window”, refers to the time range between injury and treatment during which the treatment is still effective. In this description, treatment can be a medical therapy with a fibrinolytic/thrombolytic agent (e.g., alteplase or tenecteplase), also known as i.v. thrombolysis (IVT), and its time window is in accordance with the European Stroke Organization (ESO) guidelines. Berge E et al., 2021 provides recommendations regarding time window with different thrombolytic agents, which is typically <4.5 h from stroke onset. In this description, treatment can also be an EVT (thrombectomy) which is recommended in a time window within 24 hours, particularly 8 hours after stroke onset (Jovin T G et al., 2015). It is noted that APRIL study protocol defines a time window for EVT of 6 h, with the objective of complying with said 8 h.

Neurological damage: this term as used herein refers to the neurological impairment or deterioration after suffering an ischemic stroke. Neurological impairment includes, without limitation, neuromuscular dysfunctions causing disabilities such as mobility problems, apraxia, pain syndromes, limb spasticity, and incontinence; cognitive impairment which negatively impacts patients' cognitive abilities, from memory loss to dysfunction in reasoning, speech, learning capacities, language processing and problem-solving skills; and psychiatric disturbances (emotional problems), e.g., mood disorders such as depression, anxiety, emotional instability, crisis reaction, and poststroke fatigue. This can lead to general disabilities and dependence in the daily activities of people who have suffered a stroke. In APRIL study and in this description, said damages are quantified with the infarct volume, NIHSS score and mRS score, defined hereinafter. “Neurological damage” and “neurological impairment” are used interchangeably in this description. In an aspect, ApTOLL molecules are for use in reducing the neurological damage/impairment and also for use in improving the neurological recovery after an AIS in a subject.

In an embodiment, the neurological damage comprises functional impairment and/or disability. In another embodiment, the neurological damage comprises neuromuscular disfunctions, cognitive impairment, and/or psychiatric disturbances.

In some embodiments, ApTOLL molecules are for use in the treatment of neurological damage, or in reducing the neurological damage, or in improving the neurological recovery after an ischemic stroke. In this sense, the term “neurological recovery” refers to the improvement or amelioration of the neurological damage.

NIHSS score: the National Institute of Health Stroke Scale (NIHSS) is a tool used by healthcare providers to objectively quantify the impairment caused by a stroke. The NIHSS is composed of 11 items, each of which scores a specific ability between a 0 and 4. For each item, a score of 0 typically indicates normal function in that specific ability, while a higher score is indicative of some level of impairment. The individual scores from each item are summed in order to calculate a patient's total NIHSS score. The maximum possible score is 42, with the minimum score being a 0. Score Stroke severity: 0 No stroke symptoms; 1-4 Minor stroke; 5-15 Moderate stroke; 16-20 Moderate to severe stroke; 21-42 Severe stroke (Lyden P et al., 1994). The NIHSS can help physicians quantify the severity of a stroke in the acute setting. In an embodiment, the reduction in the neurological damage and the improvement in the neurological recovery is measured by the NIHSS score.

mRS score: the modified Rankin Scale (mRS) is a commonly used scale for measuring the degree of disability or dependence in the daily activities of people who have suffered a stroke or other causes of neurological disability. The scale runs from 0-6, running from perfect health without symptoms to death: 0—No symptoms. 1—No significant disability. Able to carry out all usual activities, despite some symptoms. 2—Slight disability. Able to look after own affairs without assistance, but unable to carry out all previous activities. 3—Moderate disability. Requires some help, but able to walk unassisted. 4—Moderately severe disability. Unable to attend to own bodily needs without assistance, and unable to walk unassisted. 5—Severe disability. Requires constant nursing care and attention, bedridden, incontinent. 6—Dead (Van Swieten J C et al., 1988). In an embodiment, the reduction in the neurological damage and the improvement in the neurological recovery is measured by the mRS score.

Infarct volume: infarct volume is a direct measurement of the damaged brain tissue, one of the final pathologic steps leading to the clinical deficits caused by an ischemic stroke. Therefore, final infarct volume derived from magnetic resonance imaging (MRI) represents an objective and valid measurement of stroke consequences. In APRIL study all images were read by appropriately trained local clinicians. ASPECTS for patient selection were determined independently by the local clinicians and computed tomography perfusion (CTP) images with mismatch determination were read to determine the ischemic core at baseline. Final infarct volume (FIV) post-procedure (at 72 h) was determined by MRI-FLAIR (MRI-Fluid-Attenuated Inversion Recover) or on CT if MRI was not available.

Cerebral or brain edema: this term as used herein refers to a severe complication of AIS which is the cause of death in 5% of all patients with cerebral infarction. Cerebral edema is caused by endothelial dysfunction of the capillaries, resulting in breakdown of the blood-brain barrier (BBB). Edema causes tissue shifts and increased intracranial pressure that can cause death. In clinical practice, the edema is determined using imaging techniques, i.e., computed tomography or magnetic resonance imaging.

ASPECTS: The Alberta stroke program early CT score (ASPECTS) is a 10-point quantitative topographic CT scan score, developed to offer the reliability and utility of a standard CT examination with a reproducible grading system to assess early ischemic changes (<3 hours from symptom onset) on pretreatment CT studies in patients with AIS of the anterior circulation. This CT score is simple and reliable and identifies stroke patients unlikely to make an independent recovery despite thrombolytic treatment. The score divides the middle cerebral artery (MCA) territory into 10 regions of interest. ASPECTS is, therefore, a topographic scoring system applying a quantitative approach that does not ask physicians to estimate volumes from two-dimensional images (Pexman J H et al., 2011).

Subject: The terms “subject”, “patient”, and “individual”, and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The uses and methods described herein are applicable to both human therapy and veterinary applications. In an embodiment, the subject is a human, and particularly, a human with the characteristics described in section “Characterization of the subject” of this description.

Identity: this term as used herein refers to the overall monomer conservation between polymeric molecules, e.g., between polynucleotide molecules (e.g., DNA molecules and/or RNA molecules). The term “identical” without any additional qualifiers, e.g., nucleic acid A is identical to nucleic acid B, implies the sequences are 100% identical (100% sequence identity). Describing two sequences as, e.g., “70% identical”, is equivalent to describing them as having, e.g., “70% sequence identity”. The comparison of sequences and determination of percent identity between two sequences can be accomplished using a mathematical algorithm, such as BLAST, Needle, Stretcher, Water, Matcher, and Needleman-Wunsch among many others known in the art. Sequence alignments can be conducted using methods known in the art such as MAFFT, Clustal (ClustalW, Clustal X or Clustal Omega), MUSCLE, etc.

About: The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). As used herein, the terms “about” or “at least about” when applied to a series of values or range, apply equally to all member of the list. Accordingly, “at least about 1, 2, 3, 4 . . . ” would be interchangeable with “at least about 1, at least about 2, at least about 3, at least about 4 . . . ”.

### Clinical Outcomes

Reduction of Risk of Intracranial Hemorrhages after AIS

As discussed above, hemorrhagic complications are a common occurrence after thrombolytic therapies such as tPA or mechanical thrombectomy such as EVT, which can result in worsening functional outcomes and even death in stroke patients.

The APRIL study showed that the administration of ApTOLL (SEQ ID NO: 1) in combination with EVT in selected stroke patients limit the phenomena of intracranial hemorrhages and reperfusion damage that may occur after recanalization (EXAMPLE 1). Only 4.8% of stroke patients treated with the combination of ApTOLL and EVT developed symptomatic intracranial hemorrhages, compared to a 7.3% who were treated with EVT only (Table 4). Therefore, the risk of triggering symptomatic intracranial hemorrhages after having suffered an ischemic stroke is reduced by up to a 34.25%, an unexpected reduction that can be a major solution for AIS patients and lead to an increase in survival. Table 4 also shows that a 40.47% of patients treated with ApTOLL 0.2 mg/kg suffered an intracranial hemorrhage, in comparison to a 47.27% of patients with intracranial hemorrhages not treated with ApTOLL (placebo). Therefore, there is a reduction of 14.39% in the risk of suffering intracranial hemorrhages for patients treated with ApTOLL 0.2 mg/kg.

Accordingly, the present invention provides ApTOLL molecules for use in reducing the risk of intracranial or secondary hemorrhages (or preventing intracranial hemorrhages or reducing the occurrence of intracranial hemorrhages) after an acute ischemic stroke in a subject, wherein the ApTOLL molecules are administered after the acute ischemic stroke (i.e., after symptoms onset or stroke onset).

It is understood that the uses disclosed herein can alternatively be formulated as a method of reducing the risk of (suffering) intracranial hemorrhages (or preventing intracranial hemorrhages or reducing the occurrence of intracranial hemorrhages) after an acute ischemic stroke in a subject, the method comprising administering ApTOLL molecules to the subject, wherein the ApTOLL molecules are administered after the acute ischemic stroke.

In an embodiment, the intracranial hemorrhage is hemorrhagic transformation. In another embodiment, the intracranial hemorrhage is symptomatic. In another embodiment, the intracranial hemorrhage is asymptomatic.

In some embodiments, the reduction in the risk of intracranial hemorrhages is of between 5% and 90%, compared to control conditions, e.g., compared to the risk in a subject not treated with an ApTOLL molecule. In an embodiment, the reduction in the risk of intracranial hemorrhages is of at least about: 5%, 10%, 15%, 20%, 25%, 30%, 35%, 40%, 45%, 50%, 55%, 60%, 65%, 70%, 75%, 80%, 85%, or 90%, with respect to a subject not treated with an ApTOLL molecule. In an embodiment, the reduction in the risk of intracranial hemorrhages is of at least about: 5%, 10%, 15%, 20%, 25%, 30%, 35%, or 40% with respect to a subject not treated with an ApTOLL molecule. In a particular embodiment, the reduction in the risk of intracranial hemorrhages is of at least about: 10%, 11%, 12%, 13% or 14% with respect to a subject not treated with an ApTOLL molecule, and particularly about 14%. In an embodiment, ApTOLL molecule is administered at a dose of 0.2 mg/kg, and particularly ApTOLL molecule is ApTOLL (SEQ ID NO: 1).

In some embodiments, the reduction in the risk of symptomatic intracranial hemorrhages is of between 5% and 90%, compared to control conditions, e.g., compared to the risk in a subject not treated with an ApTOLL molecule. In an embodiment, the reduction in the risk of symptomatic intracranial hemorrhages is of at least about: 5%, 10%, 15%, 20%, 25%, 30%, 35%, 40%, 45%, 50%, 55%, 60%, 65%, 70%, 75%, 80%, 85%, or 90%, with respect to a subject not treated with an ApTOLL molecule. In a particular embodiment, the reduction in the risk of symptomatic intracranial hemorrhages is of at least about: 5%, 10%, 15%, 20%, 25%, 30%, 35%, or 40% with respect to a subject not treated with an ApTOLL molecule; particularly the reduction is of at least about: 30%, 31%, 32%, 33% or 34%, and more particularly about 34%. In an embodiment, ApTOLL molecule is administered at a dose of 0.2 mg/kg, and particularly ApTOLL molecule is ApTOLL (SEQ ID NO: 1).

As described, the primary criterion for candidate selection in reperfusion is the time from stroke symptom onset. Reperfusion therapy must be administered within a narrow window time of up to 4.5 h after stroke onset for tPA, and up to 24 h for EVT. The restriction on tPA treatment beyond 4.5 h disqualifies the majority of stroke patients admitted beyond this time-window (around 85%), thereby drastically limiting the eligible population. The timing of treatment is important, because giving a strong blood thinner like tPA during a stroke can cause bleeding inside the brain.

In APRIL study, the administration of ApTOLL together with EVT has been shown to reduce intracranial hemorrhages such as ICH and HT, i.e., treatment with tPA and EVT did not result in as much intracranial bleedings inside the brain due to the effects of ApTOLL. Therefore, ApTOLL can effectively reach the tissue at risk exerting a protective effect directly in the area of ischemic penumbra potentially extending the therapeutic window of reperfusion therapies.

Accordingly, in an embodiment, the administration of an ApTOLL molecule of the present invention (e.g., SEQ ID NO: 1) to a subject after ischemic stroke extends the therapeutic window of reperfusion therapies.

In an embodiment, the administration of an ApTOLL molecule to a subject after ischemic stroke extends the therapeutic window of pharmacological thrombolysis (e.g., tPA). In a particular embodiment, the therapeutic window of pharmacological thrombolysis (e.g., tPA) is extended to at least about: 5 hours, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h from stroke onset.

In an embodiment, the administration of an ApTOLL molecule to a subject after ischemic stroke extends the therapeutic window of EVT (e.g., thrombectomy). In a particular embodiment, the therapeutic window of EVT is extended to at least about: 25 hours, 26 h, 27 h, 28 h, 29 h, 30 h, 31 h, 32 h, 33 h, 34 h, 35 h, 36 h, 37 h, 38 h, 39 h, 40 h, 41 h, 42 h, 43, 44 h, 45 h, 46 h, 47, or 48 h from stroke onset.

Besides the reduction in secondary hemorrhages, APRIL study has also demonstrated that the administration of ApTOLL 0.2 mg/kg resulted in a reduction in mortality, a reduction in the final infarct volume, an improvement in early neurological impairment and long term disability (measured by NIHSS score and mRS score) and a reduction in brain edema.

Therefore, in some embodiments, the administration of ApTOLL molecules to a subject after ischemic stroke results in
(i) a reduction in the infarct volume;
(ii) a reduction in the NIHSS score;
(iii) an increase in the mRS score of 0-2 (i.e., the reduction in the mRS score to 0-2);
(iv) an increase in survival;
(v) a reduction in brain edema; and
(vi) any combination thereof.

The uses disclosed herein can alternatively be formulated as the provision of ApTOLL molecules (e.g., SEQ ID NO: 1) for use in a treatment (i.e., therapy or method) to reduce the risk of intracranial hemorrhages or secondary hemorrhages after an acute ischemic stroke in a subject, wherein the treatment comprises:
(a) selecting a patient within about 6 hours from the acute ischemic stroke onset,
(b) administering a dose of the aptamer to the patient of at least 0.2 mg/kg, and
(c) measuring intracranial hemorrhages or secondary hemorrhages, particularly by imaging procedures,
   wherein the administration of ApTOLL molecules reduces the risk of intracranial hemorrhages or secondary hemorrhages with respect to references values from subjects not treated with the ApTOLL molecules.
   Reduction of Risk of Intracranial Hemorrhages after a Thrombotic Disorder

As mentioned above, the reduction in intracranial hemorrhages was a result that exceeded the expectations of the APRIL study. ApTOLL (SEQ ID NO: 1) is a good candidate for reducing intracranial hemorrhages in patients having suffered an ischemic stroke. Further, evidence provided in APRIL study makes it plausible that ApTOLL could also be useful in reducing intracranial hemorrhages in similar conditions, particularly thrombotic disorders. For instance, fibrinolytics are also administered after a myocardial infarction, which can trigger systemic (remote) hemorrhagic transformation, e.g., in the digestive system or local hemorrhages. Therefore, the effects of ApTOLL in reducing intracranial hemorrhages could be plausible also for thrombotic disorders other than ischemic stroke.

Accordingly, the present invention also relates to ApTOLL molecules for use in reducing the risk of intracranial or secondary hemorrhages (or preventing intracranial hemorrhages or reducing the occurrence of intracranial hemorrhages) after a thrombotic disorder in a subject, wherein the ApTOLL molecules are administered after the thrombotic disorder.

Alternatively, the present invention also relates to a method of reducing the risk of (suffering) intracranial hemorrhages (or preventing intracranial hemorrhages or reducing the occurrence of intracranial hemorrhages) after a thrombotic disorder in a subject, the method comprising administering ApTOLL molecules to the subject, wherein the ApTOLL molecules are administered after the thrombotic disorder.

In an embodiment, the thrombotic disorder is associated with a thrombotic event, i.e., thrombosis. In an embodiment, the thrombotic disorder is a disease or disorder selected from the group consisting of arterial thrombosis including atherothrombosis, venous thrombosis, ischemic events, acute coronary artery syndrome, thrombotic occlusion of coronary arteries, coronary thrombosis, cerebrovascular accidents due to atherosclerosis (particularly thrombosis), myocardial infarction (heart attack), acute cerebrovascular ischemia (ischemic stroke), percutaneous coronary intervention, stenting thrombosis, restenosis, diseases of the aorta and its branches (such as aortic aneurysm, thrombosis), peripheral artery disease, venous thrombosis, acute phlebitis and pulmonary embolism, cancer-associated thrombosis (Trousseau syndrome), inflammatory thrombosis and thrombosis associated to infection, and deep vein thrombosis, among other embolisms.

In a particular embodiment, the thrombotic disorder is selected from the group consisting of cerebral artery diseases, cerebral vascular diseases or coronary diseases.

In a particular embodiment, the thrombotic disorder is a cerebral vascular disease (e.g., stroke or ischemia). In a particular embodiment, the cerebral vascular disease is stroke.

In a particular embodiment, the thrombotic disorder is a coronary disease. Coronary diseases include, without limitation, e.g., cardiovascular diseases including unstable angina pectoris, myocardial infarction, acute myocardial infarction, coronary artery disease, coronary revascularization, coronary restenosis, ventricular thromboembolism, atherosclerosis, coronary artery disease (e. g., arterial occlusive disorders), plaque formation, cardiac ischemia, including complications related to coronary procedures, such as percutaneous coronary artery angioplasty (balloon angioplasty) procedures.

In a particular embodiment, the thrombotic disorder is a venous thromboembolism disease, e.g., diseases involving leg swelling, pain and ulceration, pulmonary embolism, abdominal venous thrombosis. In another embodiment, the thrombotic disorder is a thrombotic microangiopathy, vascular purpura, among others.

Medical conditions associated with thrombolytics are arterial thrombosis, deep vein thrombosis, acute myocardial infarction, acute ischemic stroke, IV catheter clot, pulmonary embolism, thrombotic/thromboembolic disorder, among others.

In an embodiment, ApTOLL molecule is administered at a dose of 0.2 mg/kg, and particularly ApTOLL molecule is ApTOLL (SEQ ID NO: 1).

In an embodiment, the uses and methods described herein for reducing the risk of intracranial hemorrhages after a thrombotic disorder in a subject, further comprise the use of thrombolytics. Thrombolytic drug or agents are e.g., streptokinase, alteplase, reteplase, tenecteplase, urokinase, prourokinase, anistreplase (APSAC), among others.

### Reduction of Neurological Damage and Improvement of Neurological Recovery

The APRIL study showed consistent positive results in most predefined secondary outcome measures including final infarct volume, early neurological impairment and long-term disability (EXAMPLE 1). Of note, values of said three measures were improved at the same time in APRIL study.

Accordingly, the present invention also provides ApTOLL molecules for use in reducing the neurological damage and in improving the neurological recovery after an acute ischemic stroke in a subject, wherein the ApTOLL molecules are administered after the acute ischemic stroke.

The uses disclosed herein can alternatively be formulated as a method of reducing the neurological damage and in improving the neurological recovery after an acute ischemic stroke in a subject, the method comprising administering ApTOLL molecules to the subject, wherein the ApTOLL molecules are administered after the acute ischemic stroke.

As mentioned above, inventors found that the reduction of intracranial hemorrhages was unexpected and consequently resulted in reduced neurological damage and improved neurological recovery.

Therefore, in an embodiment, ApTOLL molecules are for use in reducing the neurological damage and in improving the neurological recovery after an acute ischemic stroke in a subject, wherein the ApTOLL molecules are administered after the acute ischemic stroke, and wherein the ApTOLL molecules reduce the risk of intracranial hemorrhage (or prevent intracranial hemorrhages) (e.g., HT and sICH); in other words, the reduction of neurological damage and improvement of neurological recovery is mediated by a reduction of the risk of intracranial hemorrhages (or a prevention of intracranial hemorrhages).

Alternatively, another embodiment relates to a method of reducing the neurological damage and improving the neurological recovery after an acute ischemic stroke in a subject, the method comprising administering ApTOLL molecules to the subject, wherein the ApTOLL molecules are administered after the acute ischemic stroke, and wherein the reduction of neurological damage and the improvement of neurological recovery is mediated by a reduction of the risk of intracranial hemorrhages.

In an embodiment, ApTOLL molecules are for use in reducing the neurological damage after an acute ischemic stroke in a subject, wherein the ApTOLL molecules are administered after the acute ischemic stroke, and wherein the ApTOLL molecules reduce the risk of intracranial hemorrhage (or prevent intracranial hemorrhages) (e.g., HT and sICH); in other words, the reduction of neurological damage is mediated by a reduction of the risk of intracranial hemorrhages (or a prevention of intracranial hemorrhages). In an embodiment, the neurological damage comprises functional impairment and/or disability. In another embodiment, the neurological damage comprises neuromuscular disfunctions, cognitive impairment, and/or psychiatric disturbances.

In another embodiment, ApTOLL molecules are for use in improving the neurological recovery after an acute ischemic stroke in a subject, wherein the ApTOLL molecules are administered after the acute ischemic stroke, and wherein the ApTOLL molecules reduce the risk of intracranial hemorrhage (or prevent intracranial hemorrhages) (e.g., HT and sICH); in other words, the improvement of neurological recovery is mediated by a reduction of the risk of intracranial hemorrhages (or a prevention of intracranial hemorrhages).

In APRIL study, the reduction in the neurological damage and the improvement in the neurological recovery were assessed by measuring some parameters at baseline, 72 hours and/or 90 days from allocation of the subject. Those parameters are the infarct volume, the NIHSS score and the mRS score.

Accordingly, in some embodiments, the reduction in the neurological damage and the improvement in the neurological recovery is measured by:
(i) the infarct volume;
(ii) the NIHSS score;
(iii) the mRS score; or
(iv) any combination thereof.

In a particular embodiment, the neurological damage is measured by the infarct volume, NIHSS score and mRS score, particularly the infarct volume. In another particular embodiment, the improvement in the neurological recovery is measured by the infarct volume, NIHSS score and mRS score, particularly the NIHSS score and mRS score, and more particularly the mRS score.

In a particular embodiment, the functional impairment is measured by the NIHSS score. In another embodiment, the disability is measured by the mRS score.

The uses disclosed herein can alternatively be formulated as the provision of ApTOLL molecules (e.g., SEQ ID NO: 1) for use in a treatment (i.e., therapy or method) to reduce the neurological damage and improve the neurological recovery after an acute ischemic stroke in a subject, wherein the treatment comprises:
(a) selecting a patient within about 6 hours from the acute ischemic stroke onset,
(b) administering a dose of the aptamer to the patient of at least 0.2 mg/kg, and
(c) measuring infarct volume, NIHSS score, mRS score, or a combination thereof,
   wherein the reduction of neurological damage and improvement of neurological recovery consists of a:
(i) reduction of the infarct volume,
(ii) reduction of the NIHSS score, and/or
(iii) reduction of the mRS score to 0-2,
   with respect to references values from subjects not treated with the ApTOLL molecules.

### Infarct Volume

In APRIL study, the infarct volume was measured by MRI/CTP at baseline, and at 72±24 hours from allocation. In those cases where MRI was not available at 72±24 h, a CT at 24 h was measured. Basal infarct volume cannot be considered as FIV in any case. In those cases where there was not any image post-procedure (which means after ApTOLL administration), the patient was considered “missing”. Reduction of infarct volume has a direct correlation with anatomical protection with functional and neurological performance in patients having suffered AIS.

Therefore, in an embodiment, the reduction in the neurological damage and the improvement in the neurological recovery is measured by the infarct volume.

ApTOLL biological impact on infarct volume was assessed through the change of infarct volume from baseline to final infarct volume at 72 h, and also through the comparison of final infarct volume at 72 hours between study groups (placebo vs. ApTOLL).

Thus, the present invention relates to ApTOLL molecules for use in reducing the neurological damage and in improving the neurological recovery after an acute ischemic stroke in a subject, wherein the ApTOLL molecules are administered after the acute ischemic stroke, and wherein the ApTOLL molecules reduce the infarct volume of the subject compared to a subject not treated with an ApTOLL molecule.

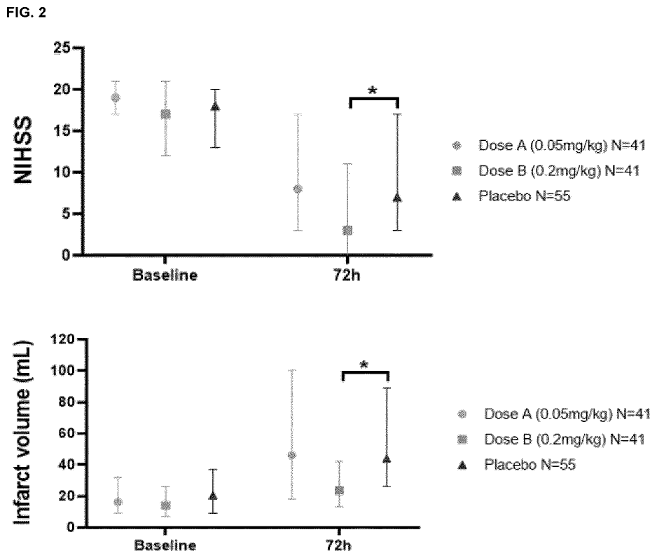

The reduction in the neurological damage and the improvement in the neurological recovery is measured by a reduction in the final infarct volume, compared to a subject not treated with an ApTOLL molecule (e.g., placebo) in a specific timepoint (e.g., 72 h from allocation). Table 4 and FIG. 2 shows that patients treated with ApTOLL 0.2 mg/kg had a median final infarct volume of 23.5 ml at 72 h, in comparison to patients not treated with ApTOLL (placebo) that had a final infarct volume of 44 ml. Therefore, patients treated with ApTOLL had a reduction of 46.6% of final infarct volume with respect to a subject not treated with ApTOLL.

Accordingly, in an embodiment, the reduction in the final infarct volume at short term, e.g., 72 hours, is of at least about: 10%, 15%, 20%, 25%, 30%, 35%, 40%, 45%, or 50% with respect to a subject not treated with an ApTOLL molecule. In a particular embodiment, the reduction in the final infarct volume at 72 h is of at least about 46% with respect to a subject not treated with an ApTOLL molecule.

In another embodiment, the final infarct volume at short term, e.g., 72 h, is between about 10 ml and about 45 ml, more particularly between 20 ml and 25 ml.

In an embodiment, ApTOLL molecule is administered at a dose of 0.2 mg/kg, and particularly ApTOLL molecule is ApTOLL (SEQ ID NO: 1).

The reduction in the neurological damage and the improvement in the neurological recovery can also be measured by a reduction in the infarct volume over time (e.g., from baseline to 72 h), compared to a subject not treated with an ApTOLL molecule (e.g., placebo). Table 5 shows that patients treated with ApTOLL 0.2 mg/kg had a baseline infarct volume of 14 ml and a final infarct volume at 72 h of 23.5 ml, thereby having an increase of 67.86% in the infarct volume caused by the evolution of the stroke. Instead, patients not treated with ApTOLL (placebo) had a baseline infarct volume of 20.50 ml and a final infarct volume at 72 h of 44 ml, thereby having an increase of 114.63% in the infarct volume. Thus, patients treated with ApTOLL resulted in a minor increase of the infarct volume in comparison to patients not treated with ApTOLL. In particular, patients not treated with ApTOLL have an increase in the infarct volume over time of 40.80% with respect to patients treated with ApTOLL.

Accordingly, in an embodiment, the reduction in the infarct volume over time, e.g., from baseline to 72 hours, is of at least about: 10%, 15%, 20%, 25%, 30%, 35%, 40%, 45%, or 50% with respect to a subject not treated with an ApTOLL molecule. In a particular embodiment, the reduction in the infarct volume over time is of at least about 40% with respect to a subject not treated with an ApTOLL molecule. In an embodiment, ApTOLL molecule is administered at a dose of 0.2 mg/kg, and particularly ApTOLL molecule is ApTOLL (SEQ ID NO: 1).

To evaluate the early clinical course and long-term neurological outcome, change from baseline of NIHSS score and mRS scale respectively was calculated in APRIL study, and also the comparison between groups at the end time (72 hours for NIHSS and 90 days for mRS).

### NIHSS Score

The NIHSS score was measured at baseline and at 72 hours post-baseline to evaluate the early clinical course. The NIHSS assessed at 72 h was 7 in patients allocated to placebo and 3 in patients allocated to ApTOLL 0.2 mg/kg (mean difference of log-transformed 72 h NIHSS vs. placebo 45%; 95% CI: −67% to −10%) (Table 4, FIG. 2). This represents a reduction of 57.14% compared to placebo.

Therefore, in an embodiment, the reduction in the neurological damage and the improvement in the neurological recovery is measured by the NIHSS score. In a particular embodiment, the neurological damage is a functional impairment.

Thus, the present invention relates to ApTOLL molecules for use in reducing the neurological damage and in improving the neurological recovery after an acute ischemic stroke in a subject, wherein the ApTOLL molecules are administered after the acute ischemic stroke, and wherein the ApTOLL molecules reduce the NIHSS score of the subject compared to a subject not treated with an ApTOLL molecule.

In an embodiment, the reduction in the neurological damage and the improvement in the neurological recovery is measured by a reduction in the NIHSS score at short term (e.g., 72 h), compared to a subject not treated with an ApTOLL molecule (e.g., placebo). In an embodiment the reduction in the NIHSS score is between 5% and 90%, with respect to a subject not treated with an ApTOLL molecule. In an embodiment, the reduction in the NIHSS score is of at least about: 5%, 10%, 15%, 20%, 25%, 30%, 35%, 40%, 45%, 50%, 55%, 60%, 65%, 70%, 75%, 80%, 85% or 90%, with respect to a subject not treated with an ApTOLL molecule. In a particular embodiment, the reduction in the NIHSS score is of at least about: 40%, 45%, 50%, 55% or 60%, with respect to a subject not treated with an ApTOLL molecule, particularly the reduction is of at least about: 50%, 51%, 52%, 53%, 54%, 55%, 56% or 57%, and more particularly about 57%. In an embodiment, ApTOLL molecule is administered at a dose of 0.2 mg/kg, and particularly ApTOLL molecule is ApTOLL (SEQ ID NO: 1).

### mRS Score

In APRIL study, the mRS scale was measured at baseline (pre-stroke mRS), and 72 h and 90 days post-baseline to evaluate the long-term neurological outcome. In those cases where 72 h or 90 d data was missing, the last measure post-procedure (which means after ApTOLL administration) was considered.

Therefore, in an embodiment, the reduction in the neurological damage and the improvement in the neurological recovery is measured by the mRS score. In a particular embodiment, the neurological damage is a disability.

Thus, the present invention relates to ApTOLL molecules for use in reducing the neurological damage and in improving the neurological recovery after an acute ischemic stroke in a subject, wherein the ApTOLL molecules are administered after the acute ischemic stroke, and wherein the ApTOLL molecules improve the mRS score of the subject compared to a subject not treated with an ApTOLL molecule, i.e., reduces the mRS score of 3-6 and/or increases the mRS score of 0-2 (i.e., reduces the mRS score to 0-2). The improvement of mRS score is reflected by a decrease in the mRS score, aiming for the lower values between 0-2.

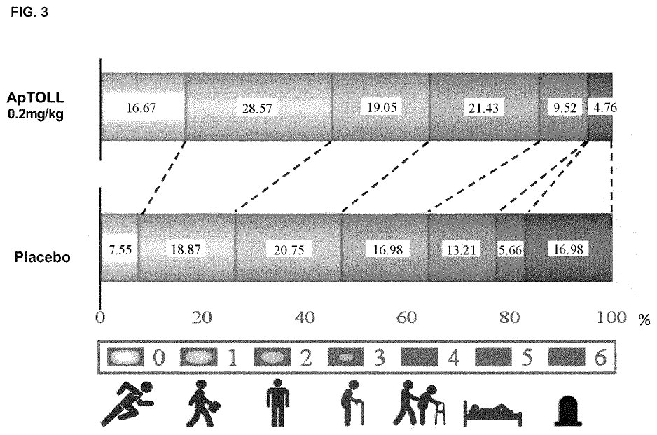

The reduction in the neurological damage and the improvement in the neurological recovery is measured by a reduction in the mRS score of 3-6 or by an increase in the mRS score of 0-2 (i.e., by a reduction in the mRS score to 0-2), compared to a subject not treated with an ApTOLL molecule (e.g., placebo) in a specific timepoint (e.g., 90 days). Table 4 and FIG. 3 show that patients treated with ApTOLL 0.2 mg/kg had a reduction in the mRS score of 3-6 (from moderate-severe disabilities to death) of 32.40% with respect to a subject not treated with ApTOLL, i.e., there are 32.40% fewer patients in mRS score of 3-6 allocated to ApTOLL 0.2 mg/kg group. Further, Table 4 and FIG. 3 also show that patients treated with ApTOLL 0.2 mg/kg had an increase in the mRS score of 0-2 (from no symptoms 0 to slight disability 2) of 36.29% with respect to a subject not treated with ApTOLL, i.e., there are 36.29% more patients in mRS score of 0-2 allocated to ApTOLL 0.2 mg/kg group (meaning that the mRS score was reduced—from a higher mRS value—to 0-2); and particularly patients treated with ApTOLL 0.2 mg/kg had an increase in the mRS score of 0-1 (from no symptoms 0 to no significant disability 1) of 71.23% with respect to a subject not treated with ApTOLL (meaning that the mRS score was reduced—from a higher mRS value—to 0-2). This result suggests that patients treated with ApTOLL within 6 hours from stroke onset had milder disabilities at long-term (90 days), with more patients in the mRS score of 0-1 and 0-2 and fewer in the mRS score of 3-6, compared to placebo group.

Accordingly, in an embodiment, the reduction in the mRS score of 3-6 at long-term, e.g., 90 days, is of at least about: 10%, 15%, 20%, 25%, 30%, or 35% with respect to a subject not treated with an ApTOLL molecule. In a particular embodiment, the reduction in the mRS score of 3-6 at 90 days is of at least about 32% with respect to a subject not treated with an ApTOLL molecule.

In another embodiment, the increase in the mRS score of 0-2 (i.e., reduction in the mRS score to 0-2) at long-term, e.g., 90 days, is of at least about: 10%, 15%, 20%, 25%, 30%, 35%, or 40% with respect to a subject not treated with an ApTOLL molecule. In a particular embodiment, the increase in the mRS score of 0-2 at 90 days is of at least about 36% with respect to a subject not treated with an ApTOLL molecule.

In another embodiment, the increase in the mRS score of 0-1 (i.e., reduction in the mRS score to 0-1) at long-term, e.g., 90 days, is of at least about: 40%, 45%, 50%, 55%, 60%, 65%, 70% or 75% with respect to a subject not treated with an ApTOLL molecule. In a particular embodiment, the increase in the mRS score of 0-1 at 90 days is of at least about 71% with respect to a subject not treated with an ApTOLL molecule.

In an embodiment, the increase in the mRS score of 0 (i.e., reduction in the mRS score to 0) at 90 days is of at least about: 50%, 55%, 60%, 65%, 70%, 75%, 80%, 85%, 90%, 95%, 100%, 105%, 110%, 115%, or 120% with respect to a subject not treated with an ApTOLL molecule. In a particular embodiment, the increase in the mRS score of 0 at 90 days is of at least about 120% with respect to a subject not treated with an ApTOLL molecule.

In another embodiment, the increase in the mRS score of 1 (i.e., reduction in the mRS score to 1) at 90 days is of at least about: 30%, 35%, 40%, 45%, or 50% with respect to a subject not treated with an ApTOLL molecule. In a particular embodiment, the increase in the mRS score of 1 at 90 days is of at least about 50% with respect to a subject not treated with an ApTOLL molecule.

In another embodiment, the reduction in the mRS score of 4 at 90 days is of at least about: 10%, 15%, 20%, or 25% with respect to a subject not treated with an ApTOLL molecule. In a particular embodiment, the reduction in the mRS score of 4 at 90 days is of at least about 25% with respect to a subject not treated with an ApTOLL molecule.

In another embodiment, the reduction in the mRS score of 5-6 at 90 days is of at least about: 40%, 45%, 50%, 60%, 65%, 70%, 75%, or 80% with respect to a subject not treated with an ApTOLL molecule. In a particular embodiment, the reduction in the mRS score of 5-6 at 90 days is of at least about 75% with respect to a subject not treated with an ApTOLL molecule.

In an embodiment, ApTOLL molecule is administered at a dose of 0.2 mg/kg, and particularly ApTOLL molecule is ApTOLL (SEQ ID NO: 1).

The reduction in the neurological damage and the improvement in the neurological recovery can also be measured by an increase in the mRS score of 0-2 over time (e.g., from baseline—pre-stroke mRS—to 90 days), compared to a subject not treated with an ApTOLL molecule (e.g., placebo). Table 5 shows that 100% of patients treated with ApTOLL 0.2 mg/kg had a pre-stroke mRS of 0-2, which decreased to 64.29% at 90 days, representing a 35.71% decrease. In contrast, 98.15% of patients not treated with ApTOLL (placebo) had a pre-stroke mRS of 0-2, which decreased to 47.17% at 90 days, representing a 51.94% decrease. Thus, ApTOLL-treated patients have a smaller reduction in mRS score of 0-2 compared to patients not treated with ApTOLL. In particular, patients not treated with ApTOLL have a reduction in the mRS score of 0-2 over time of 31.25% with respect to patients treated with ApTOLL.

Accordingly, in an embodiment, the increase in the mRS of 0-2 (i.e., reduction in the mRS to 0-2) over time, e.g., from baseline to 90 days, is of at least about: 10%, 15%, 20%, 25%, 30%, 35%, 40%, 45%, or 50% with respect to a subject not treated with an ApTOLL molecule. In a particular embodiment, the increase in the mRS of 0-2 over time is of at least about 31% with respect to a subject not treated with an ApTOLL molecule. In an embodiment, ApTOLL molecule is administered at a dose of 0.2 mg/kg, and particularly ApTOLL molecule is ApTOLL (SEQ ID NO: 1).

In a particular embodiment, the reduction in the neurological damage and the improvement in the neurological recovery are measured by: (i) the reduction in the infarct volume; (ii) the reduction in the NIHSS score; and/or (iii) the reduction in the mRS score of 3-6 and/or the increase in the mRS score of 0-2 (i.e., reduction in the mRS to 0-2); compared to a subject not treated with an ApTOLL molecule (e.g., placebo). Particularly, the reduction in the neurological damage and the improvement in the neurological recovery are measured by: (i) the reduction in the infarct volume; (ii) the reduction in the NIHSS score; and (iii) the increase in the mRS score of 0-2 (i.e., reduction in the mRS to 0-2).

Thus, the present invention relates to ApTOLL molecules for use in reducing the neurological damage and in improving the neurological recovery after an acute ischemic stroke in a subject, wherein the ApTOLL molecules are administered after the acute ischemic stroke, and wherein the ApTOLL molecules (i) reduce the infarct volume, (ii) reduce the NIHSS score, and/or (iii) increase the mRS score of 0-2 of the subject (i.e., reduction in the mRS to 0-2) or decrease the mRS score of 3-6, compared to a subject not treated with an ApTOLL molecule. Particularly, the ApTOLL molecules (i) reduce the infarct volume, (ii) reduce the NIHSS score, and (iii) increase the mRS score of 0-2 of the subject (i.e., reduction in the mRS to 0-2).

In a particular embodiment: (i) the reduction of infarct volume at 72 hours is between about 10% and about 50%, more particularly of at least about 40%; (ii) the reduction of NIHSS is between about 40% and about 60%, more particularly of at least about 55%; and/or (ii) the increase of mRS 0-2 (i.e., reduction in the mRS to 0-2) is between about 10% and about 40%, more particularly of at least about 35%, compared to a subject not treated with an ApTOLL molecule.

As explained above, in APRIL study the reduction in the neurological damage and the improvement in the neurological recovery were assessed by measuring the infarct volume, the NIHSS score and the mRS score. However, the reduction in the neurological damage and the improvement in the neurological recovery can also be assessed by other scores known by the skilled in the art, e.g., by measuring the Barthel index/score (BI), the Asian stroke disability scale (ASDS), the Glasgow Outcome Score (GOS), among others.

### Increase in Survival and Reduction of Brain Edema

The APRIL study showed that the administration of ApTOLL in combination with EVT in selected stroke patients is safe and reduces the mortality at 90 days. Moreover, the efficacy of ApTOLL as a neuroprotective drug in acute cerebral ischemia is supported by consistent positive results in most predefined primary outcome measures including reduction of brain edema (EXAMPLE 1, 1.13-1.15, Table 4).

Accordingly, the present invention also provides ApTOLL molecules for use in increasing survival and/or reducing brain edema after an acute ischemic stroke in a subject, wherein the ApTOLL molecules are administered after the acute ischemic stroke.

It is understood that the uses disclosed herein can alternatively be formulated as a method of increasing survival and/or reducing brain edema after an acute ischemic stroke in a subject, the method comprising administering ApTOLL molecules to the subject, wherein the ApTOLL molecules are administered after the acute ischemic stroke.

As mentioned above, inventors found that the reduction of intracranial hemorrhages was unexpected and resulted in increased survival, and reduced brain edema.

Therefore, in an embodiment, ApTOLL molecules are for use in increasing survival and/or reducing brain edema after an acute ischemic stroke in a subject, wherein the ApTOLL molecules are administered after the acute ischemic stroke, and wherein the ApTOLL molecules reduce the risk of intracranial hemorrhages (or prevent intracranial hemorrhages) (e.g., HT and sICH); in other words, the increase in survival and the reduction of brain edema is mediated by a reduction of the risk of intracranial hemorrhages.

Alternatively, another embodiment relates to a method of increasing survival and/or reducing brain edema after an acute ischemic stroke in a subject, the method comprising administering ApTOLL molecules to the subject, wherein the ApTOLL molecules are administered after the acute ischemic stroke, and wherein the increase in survival and the reduction of brain edema is mediated by a reduction of the risk of intracranial hemorrhages.

Table 4 shows that in APRIL study, deaths were reduced from 18.2% to 4.85% when administered 0.2 mg/kg ApTOLL compared to placebo, representing a reduction of deaths of 73.63%. In an embodiment, the administration of ApTOLL molecules causes a reduction in deaths with respect to placebo group. Particularly, the reduction of deaths is of at least about 5%, 10%, 15%, 20%, 25%, 30%, 35%, 40%, 45%, 50%, 55%, 60%, 65%, 70% or 75% with respect to placebo group. Particularly, the reduction of deaths is of at least about 70%, 71%, 72%, 73% or 74% with respect to placebo group, and more particularly about 73%. In an embodiment, ApTOLL molecules are administered at a dose of 0.2 mg/kg, and particularly ApTOLL molecules are ApTOLL (SEQ ID NO: 1).

In other words, APRIL study achieved a survival along the study of 95.2% when administered 0.2 mg/kg ApTOLL, and of 81.8% without ApTOLL treatment, representing an increase in survival of 14.08%. In an embodiment, the administration of ApTOLL molecules causes an increase in survival with respect to placebo group. Particularly, the increase in survival is of at least about 5%, 6%, 7%, 8%, 9%, 10%, 11%, 12%, 13% or 14% with respect to placebo group. Particularly, the increase in survival is of at least about 10% with respect to placebo group, and more particularly about 14%. In an embodiment, ApTOLL molecules are administered at a dose of 0.2 mg/kg, and particularly ApTOLL molecules are ApTOLL (SEQ ID NO: 1).

Moreover, Table 4 shows that a 2.38% of patients treated with ApTOLL 0.2 mg/kg suffered brain edema, in comparison to a 7.3% of patients with brain edema not treated with ApTOLL (placebo). Therefore, there is a reduction of 67.40% in brain edema for patients treated with ApTOLL 0.2 mg/kg. In an embodiment, the administration of ApTOLL molecules causes a reduction of brain edema with respect to placebo group. Particularly, the reduction of brain edema is of at least about 5%, 10%, 15%, 20%, 25%, 30%, 35%, 40%, 45%, 50%, 55%, 60%, 65%, or 70% with respect to placebo group. Particularly, the reduction of brain edema is of at least about 40%, 45%, 50%, 55%, 60%, 65%, 70%, 75% or 80% with respect to placebo group, and more particularly about 67%. In an embodiment, ApTOLL molecules are administered at a dose of 0.2 mg/kg, and particularly ApTOLL molecules are ApTOLL (SEQ ID NO: 1).

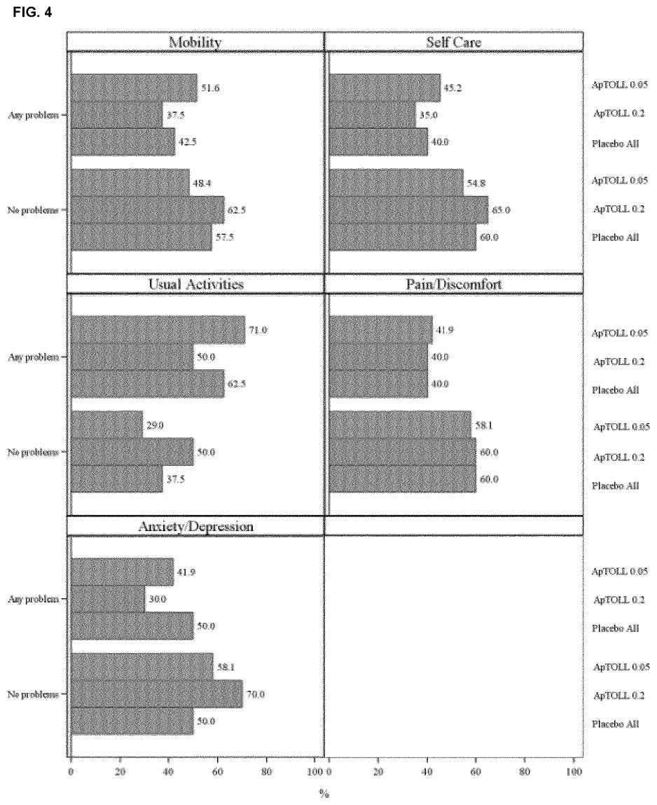

FIG. 4 is a graphical representation of the quality-of-life evaluation of patients after treatment with ApTOLL 0.05 mg/kg, 0.02 mg/kg or placebo. Quality of life evaluation includes the following criteria: mobility, self care, usual activities, pain/discomfort, and anxiety/depression. In APRIL study, it was observed an improvement in anxiety/depression complications, the performance of usual activities, mobility and self care with the administration of 0.2 mg/kg of ApTOLL compared to placebo.

Accordingly, other aspects of the invention relate to ApTOLL molecules for use in improving anxiety/depression complications, the performance of usual activities, mobility and/or self care after an acute ischemic stroke in a subject, wherein the ApTOLL molecules are administered after the acute ischemic stroke.

It is understood that the uses disclosed herein can alternatively be formulated as a method of improving anxiety/depression complications, the performance of usual activities, mobility and/or self care after an acute ischemic stroke in a subject, the method comprising administering ApTOLL molecules to the subject, wherein the ApTOLL molecules are administered after the acute ischemic stroke.

### Administration and Doses of ApTOLL Molecules

As detailed in EXAMPLE 1, APRIL study was conducted in two parts, Phase Ib and Phase IIa. During the Phase Ib, four ascending dose levels of SEQ ID NO: 1 (0.025, 0.05, 0.1 and 0.2 mg/kg) were administered, whereas two doses of SEQ ID NO: 1 (dose A=0.05 mg/kg and dose B=0.2 mg/kg) were administered during Phase IIa. Results of Phase IIa (EXAMPLE 1, 1.13-1.15) show that dose B of 0.2 mg/kg is effective in patients having suffered AIS, in comparison to the placebo and dose A groups of patients.

Therefore, in some embodiments, the ApTOLL molecules of the present invention (e.g., SEQ ID NO: 1) are administered at a dose of at least about the following values: 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.35 mg/kg, 0.4 mg/kg, 0.45 mg/kg, 0.5 mg/kg, or at least about 1 mg/kg. In a particular embodiment, the ApTOLL molecules are administered at a dose of at least about 0.2 mg/kg, particularly about 0.2 mg/kg. Even more particularly, SEQ ID NO: 1 is administered at a dose of at least about 0.2 mg/kg, particularly about 0.2 mg/kg.

In some embodiments, the ApTOLL molecules (e.g., SEQ ID NO: 1) are administered at a dose range between about 0.05 mg/kg and about 200 mg/kg. In another embodiment, the dose range is between about 0.06 mg/kg and about 50 mg/kg. In another embodiment, the dose range is between about 0.06 mg/kg and about 10 mg/kg. In another embodiment, the dose range is between about 0.1 mg/kg and about 5 mg/kg. Particularly, the dose range is between about 0.1 mg/kg and about 1 mg/kg. Particularly, the dose range is between about 0.15 mg/kg and about 1 mg/kg. Particularly, the dose range is between about 0.2 mg/kg and about 1 mg/kg. Particularly, the dose range is between about 0.2 mg/kg and about 0.5 mg/kg.

The amount of a standard single dose, considering a dose range between about 0.1 mg/kg and about 1 mg/kg, and considering a standard weight of the human subject of about 70 kg, is between about 7 mg/dose and about 70 mg/dose. Particularly, the ApTOLL molecules are administered at a dosage of at least about 14 mg/dose, particularly about 14 mg/dose. Even more particularly, SEQ ID NO: 1 is administered at a dosage of at least about 14 mg/dose, particularly about 14 mg/dose.

In some embodiments, the ApTOLL molecules (e.g., SEQ ID NO: 1) can be administered via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. In a particular embodiment, the ApTOLL molecules (e.g., SEQ ID NO: 1) are administered intravenously or intraarterially, e.g., via infusion or via bolus.

In a more particular embodiment, the ApTOLL molecules (e.g., SEQ ID NO: 1) are administered intravenously by infusion. In a particular embodiment, the infusion has a duration of about: 5 minutes, 10 min, 15 min, 20 min, 25 min, 30 min, 35 min, 40 min, or 60 min, and particularly of about 30 min.

In some embodiments, the ApTOLL molecules (e.g., SEQ ID NO: 1) are administered less than 24 hours since the ischemic stroke event. In an embodiment, the ApTOLL molecules are administered less than about: 1 hour, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h or less than about 24 h after the ischemic stroke event.

In a particular embodiment, the ApTOLL molecules (e.g., SEQ ID NO: 1) are administered within about 8 hours, particularly within about 6 hours from stroke onset. In APRIL study, patients had a median duration of stroke onset to study ApTOLL administration of 210 minutes (3.5 h). Thus, in a particular embodiment, the ApTOLL molecules are administered within about 4 h from stroke onset, and more particularly within about 3.5 h.

In APRIL study, the intravenous administration of ApTOLL (SEQ ID NO: 1) was administered together with EVT and pharmacological thrombolysis when indicated in the AIS target population. Generally, ApTOLL was administered before the EVT and after the thrombolysis, when indicated. The median time from ApTOLL infusion to recanalization (when achieved) was approximately 180 minutes (3 h).

Accordingly, in some embodiments, the ApTOLL molecules (e.g., SEQ ID NO: 1) are administered prior to the EVT (e.g., thrombectomy). In some embodiments, the ApTOLL molecules are administered about 6 hours prior to the EVT. In other embodiments, the ApTOLL molecules are administered about: 6 hours, 5.5 h, 5 h, 4.5 h, 4 h, 3.5 h, 3 h, 2.5 h, 2 h, 90 min, 60 min, 45 min, 30 min, 20 min, 10 min or about 5 min prior to the EVT. In an embodiment, the ApTOLL molecules are administered about 4 h prior to the EVT, particularly about 3 h prior to the EVT.

In other embodiments, the ApTOLL molecules (e.g., SEQ ID NO: 1) are administered concurrently with the EVT.

In other embodiments, the ApTOLL molecules (e.g., SEQ ID NO: 1) are administered after the EVT. In an embodiment, the ApTOLL molecules are administered immediately after the EVT. In an embodiment, the ApTOLL molecules are administered about: 5 min, 10 min, 15 min, 20 min, 25 min, or 30 min after the EVT.

In other embodiments, the ApTOLL molecules (e.g., SEQ ID NO: 1) are administered prior and concurrently with the EVT. In other embodiments, the ApTOLL molecules are administered prior and immediately after the EVT. In an embodiment, the ApTOLL molecules are administered at least about 20 min prior to the EVT and about 10 min after the EVT.

In APRIL study, when indicated, patients were administered with pharmacological thrombolysis (mainly with alteplase but some with tenecteplase). Administration of alteplase was via a bolus of 1-2 minutes plus an infusion of 60 minutes. Administration of tenecteplase was via a bolus of 1-2 minutes. Generally, ApTOLL was administered after tPA administration, when indicated. Since the administration of alteplase lasts longer, this could coincide with the administration of ApTOLL.

In some embodiments, the ApTOLL molecules (e.g., SEQ ID NO: 1) are administered after the pharmacological thrombolysis (e.g., tPA administration). In an embodiment, the ApTOLL molecules are administered immediately after the thrombolysis. In an embodiment, the ApTOLL molecules are administered about: 5 min, 10 min, 15 min, 20 min, 25 min, or 30 min after the thrombolysis. In another embodiment, the ApTOLL molecules are administered between about 5 min and about 30 min after the thrombolysis.

In other embodiments, the ApTOLL molecules (e.g., SEQ ID NO: 1) are administered concurrently with the pharmacological thrombolysis.

In other embodiments, the ApTOLL molecules (e.g., SEQ ID NO: 1) are administered prior and/or concurrently with the pharmacological thrombolysis. In other embodiments, the ApTOLL molecules (e.g., SEQ ID NO: 1) are administered concurrently and/or after the thrombolysis. In other embodiments, the ApTOLL molecules (e.g., SEQ ID NO: 1) are administered prior and immediately after the thrombolysis. In an embodiment, the ApTOLL molecules are administered at least about 20 min prior to the thrombolysis and about 10 min after the thrombolysis.

In some embodiments, the ApTOLL molecules (e.g., SEQ ID NO: 1) are administered after the pharmacological thrombolysis (e.g., tPA administration) and prior to the EVT (e.g., thrombectomy). In an embodiment, the ApTOLL molecules are administered between about 5 min and about 30 min after the thrombolysis and between about 4 hours and about 5 min prior to the EVT.

In other embodiments, the ApTOLL molecules (e.g., SEQ ID NO: 1) are administered concurrently and/or after the pharmacological thrombolysis and prior and/or concurrently with the EVT. In an embodiment, the ApTOLL molecules are administered concurrently and immediately after the thrombolysis and about 4 hours and about 5 min prior and/or concurrently with the EVT.

In some embodiments, the ApTOLL molecules (e.g., SEQ ID NO: 1) are administered in multiple doses. In an embodiment, the ApTOLL molecules are administered in one, two, three, four, five, six, seven, eight, nine or ten doses.

### Artery Recanalization Techniques

The uses and methods described herein further comprise additional pharmacological and/or surgical steps, e.g., administering an additional ischemic stroke treatment or thrombotic disorders treatment.

Accordingly, the ApTOLL molecules of the present invention are administered concurrently, prior, or after additional pharmacological and/or surgical steps, particularly artery recanalization. Particularly, the ApTOLL molecules are administered in combination with artery recanalization.

Artery recanalization can be induced mechanically (e.g., endovascular thrombectomy), pharmacologically (e.g., thrombolysis), or a combination thereof. In an embodiment, the artery recanalization is mechanical, pharmacological, pharmacomechanical, or a combination thereof.

In an embodiment, artery recanalization is mechanical. In a particular embodiment, the mechanical artery recanalization is an endovascular treatment (i.e., EVT). In a particular embodiment, EVT is selected from the group consisting of stent-retriever thrombectomy, balloon embolectomy, direct aspiration thrombectomy, surgical embolectomy, or a combination thereof.

In a particular embodiment, the EVT is performed within 24 hours from ischemic stroke onset. Particularly, the EVT is performed within 8 hours from stroke onset. More particularly, the EVT is performed within 6 hours from stroke onset.

In APRIL study, the quality of reperfusion after EVT was assessed with the expanded Thrombolysis in Cerebral Ischemia (eTICI) scale. Recanalization status was derived for every patient: (a) vessel recanalization after successful thrombectomy (TICI 2b or 3); (b) persistent LVO in patients with failed recanalization after EVT (TICI 0-2a). An 87% of recanalization rate was obtained. In an embodiment, the final eTICI is between 2b and 3 score. In an embodiment, the recanalization rate is at least about 87%.

In an embodiment, artery recanalization is pharmacological. In a particular embodiment, the pharmacological artery recanalization is pharmacological thrombolysis. In a particular embodiment, the pharmacological thrombolysis is a fibrinolytic therapy. In a particular embodiment, the fibrinolytic is tissue plasminogen activator (tPA, alteplase) and modified alteplase (e.g., tenecteplase).

In a particular embodiment, the pharmacological thrombolysis (e.g., tPA) is administered within 4.5 hours from stroke onset.

In an embodiment, artery recanalization is pharmacomechanical. In a particular embodiment, pharmacomechanical artery recanalization comprises EVT and pharmacological thrombolysis (e.g., tPA).

In some embodiments, the uses and methods described herein further comprise the use of imaging procedures, e.g., non-contrast computerized tomography (NCCT), computerized tomography angiography (CTA), computerized tomography perfusion (CTP) and magnetic resonance imaging (MRI) images. The following imaging and angiography variables can be extracted from imaging procedures, using the published definitions and standards: ASPECTS, hemorrhage, hemorrhage (Heidelberg), ischemic core (post hoc DWI or CTP rCBF<30% volumes when available), Tmax>6 sec volume (if CTP or PWI available), baseline occlusive lesion location, CTA collateral score (based on availability-post hoc), presence of stenosis proximal to arterial occlusive lesion, arterial occlusive lesion, collateral flow grade—ASITN, eTICI on each device pass, distal emboli, emboli to new territories, dissection, vessel perforation, final infarct volume post hoc DWI or CTP rCBF<30% volumes when available, among others. The imaging procedures can be performed at pre-baseline (drip and ship hospital), at baseline, during procedure, post-procedure (at 24 hours), post-procedure (at 72 hours) or during post-procedure course.

### Characterization of the Subject

In the APRIL study, patients with moderate infarct volumes (IV), a closed cerebral artery and with a disabling ischemic stroke at stroke onset (measured by the NIHSS score, e.g., between 5 and 25) but functionally independent before the stroke event (defined by a pre-stroke mRS score e.g., between 0 and 2), were selected to maximize the effects of ApTOLL (SEQ ID NO: 1). Thus, different criteria were used to select the subjects for treatment with ApTOLL, such as the age, the baseline NIHSS, the pre-stroke mRS score, the infarct volume at stroke onset, and the occlusion site, among others. Nevertheless, these values are not restrictive, and more patients could be eligible for treatment with an ApTOLL molecule of the present invention. All inclusion parameters were measured at stroke/symptoms onset.

In some embodiments, the subject is a human subject. In an embodiment, the subject is a man. In another embodiment, the subject is a non-pregnant woman.

In some embodiments, the subject is between about 18 and about 90 years of age. In a particular embodiment, the subject is between about 60 and about 80 years of age. In a particular embodiment, the subject is >70 years, or about 70 years of age. In a particular embodiment, the subject is about 75 years of age.

A criterion used for the selection of the subject for treatment with an ApTOLL molecule (e.g., SEQ ID NO: 1) is the baseline NIHSS, used to quantify the severity of a stroke in the acute setting. In some embodiments, the subject has a baseline NIHSS of between about 5 points and about 25 points. In some embodiments, the subject has a baseline NIHSS of between about 8 points and about 25 points. In an embodiment, the subject has a baseline NIHSS of about 16 points. In an embodiment, the subject has a baseline NIHSS between about 11 points and about 21 points. In an embodiment, the subject has a severe baseline NIHSS, i.e., between about 15 points and about 24 points. In a particular embodiment, the subject has a baseline NIHSS between about 15 points and about 20 points, particularly between about 15 points and about 18 points.

Another criterion used for the selection of the subject for treatment with an ApTOLL molecule (e.g., SEQ ID NO: 1) is the pre-stroke mRS score used to determine the neurologic disability, that is, the mRS score of subjects before the ischemic stroke event. In some embodiments, the subject has a pre-stroke mRS score of between 0 points and about 2 points.

In some embodiments, the criterion used for the selection of the subject for treatment with an ApTOLL molecule (e.g., SEQ ID NO: 1) is the time from the onset of symptoms. Accordingly, in some embodiments, the subject is selected for treatment if the onset of symptoms is less than about: 6 hours, 5 hours, 4 hours, 3 hours, 2 hours, or less than about 1 hour from the onset of the ischemic condition.

In some embodiments, the criterion used for the selection of the subject for treatment with an ApTOLL molecule (e.g., SEQ ID NO: 1) is whether the subject is a candidate to receive EVT, e.g., a thrombectomy, with or without thrombolysis (e.g., i.v. tPA). In an embodiment, the indication of EVT is based on NCCT findings (i.e., Alberta Stroke Program Early CT score, ASPECTS of 6-10). In an embodiment, the subject has an ASPECTS between about 6 and about 10.

In some embodiments, the criterion used for the selection of the subject for treatment with an ApTOLL molecule (e.g., SEQ ID NO: 1) is the presence of a single LVO (TICI 0 or TICI 1 flow) at the level of the TICA or the M1 or M2 segments of the middle cerebral artery at stroke or symptoms onset.

Another criterion used for the selection of the subject for treatment with an ApTOLL molecule (e.g., SEQ ID NO: 1) is the infarct volume. In some embodiments, the subject has a moderate infarct volume. In some embodiments, the subject has an infarct volume of between about 5 cc and about 70 cc at stroke onset or at allocation. In a particular embodiment, the subject has an infarct volume on CT perfusion (CTP) defined as cerebral blood flow (CBF)<30% between about 5 ml and about 70 ml, measured by an automated software (RAPID® software).

In some embodiments, the criterion used for the selection of the subject for treatment with an ApTOLL molecule (e.g., SEQ ID NO: 1) is blood vessel occlusion suitable for mechanical thrombectomy, e.g., determined or confirmed by Computerized Tomography Angiography (CTA). In an embodiment, the criterion used for of the subject for treatment with an ApTOLL molecule is LVO, suitable for mechanical thrombectomy as determined or confirmed by neuroimaging criteria (CT or MRI), such as:
(i) Magnetic resonance imaging (MRI) criterion: volume of diffusion-weighted imaging (DWI) restriction≥about 5 mL and ≤about 70 mL as determined, e.g., by RAPID® software; and/or,
(ii) Computerized tomography (CT) criterion: Alberta Stroke Program Early CT Score (ASPECTS) about 6 to about 10 and infarct score determined on admission cerebral blood flow (CBF)<30% and ≥about 5 mL and ≤about 70 mL determined, e.g., by RAPID® software.

In an embodiment, the subject has a volume of DWI restriction between about 5 mL and about 70 mL. In another embodiment, the subject has an ASPECTS between about 6 and about 10.

In an embodiment, the subject:
i) is between about 18 and about 90 years of age;
ii) has a baseline NIHSS of between about 8 points and about 25 points;
iii) has a pre-stroke mRS score of between 0 points and about 2 points;
iv) has an infarct volume of between about 5 cc and about 70 cc;
v) has an onset of symptoms of less than about 6 hours;
vi) is a candidate to receive EVT treatment;
vii) has an occlusion in the TICA, M1 or M2 segments of the middle cerebral artery;
viii) has a mTICI score of 0 or 1;
ix) has a volume of DWI restriction between about 5 mL and about 70 mL;
x) has an ASPECTS between about 6 and about 10; or
xi) any combination thereof,
   at stroke onset.

In a particular embodiment, the subject has a baseline NIHSS of between about 8 points and about 25 points, a pre-stroke mRS score of between 0 points and about 2 points and an infarct volume of between about 5 cc and about 70 cc at stroke onset or at allocation.

In a particular embodiment, the subject has a single LVO at the level of the TICA or the M1 or M2 segments of the middle cerebral artery and an infarct core volume on CTP defined as Cerebral

Blood Flow<30% between about 5 ml and about 70 ml measures by an automated software (RAPID®, iSchemaView).

In a particular embodiment, the subject is between about 18 and about 90 years of age, has a LVO within a 6 h window, has an ASPECTS between about 6 and about 10 and estimated infarct core volume on CT-perfusion between about 5 ml and about 70 ml.

In APRIL study, a greater effect has been observed on the final infarct volume and on long-term functional improvement (mRS) in the population over 70 years of age with the administration of 0.2 mg/kg ApTOLL. Accordingly, in one embodiment, the subject is >70 years of age. It also seems that there is a greater effect in patients with baseline NIHSS of more than 15 points on the reduction of infarct volume and on mRS score with the administration of 0.2 mg/kg ApTOLL. Accordingly, in one embodiment, the subject has a baseline NIHSS>15 points.

### ApTOLL Molecules

The term “ApTOLL molecule” as used herein refers to an aptamer selected from SEQ ID NO: 1-24 or SEQ ID NO: 1-16, a variant and/or a derivative of said aptamers, or a chemically modified aptamer thereof. The APTOLL molecules have the capability of binding specifically to at least one epitope located on the extracellular domain of TLR-4 and inhibiting TLR-4. The particularities and features of all these aptamers are disclosed in WO2015197706A1, WO2020230108A1 and WO2020230109A1 (AptaTargets SL), which are herein incorporated by reference in their entirety.

The aptamers of SEQ ID NO: 1-16 have lengths between 45 nucleotides to 78 nucleotides. The aptamers of SEQ ID NO: 17-22 are variants (mutants) of ApTOLL sequence (SEQ ID NO: 1), and SEQ ID NO: 23 and 24 are mutants of 4F aptamer (SEQ ID NO: 4). SEQ ID NO: 4 is 100% identical to SEQ ID NO: 1 in its central region and has 5′ end and 3′ end extensions with respect to SEQ ID NO: 1. EXAMPLE 3 shows that all these variants with different percentage of sequence identity and/or extension at the 5′ end and 3′ end of ApTOLL (SEQ ID NO: 1) have TLR4 antagonist activity comparable to that of ApTOLL (SEQ ID NO: 1).

In some embodiments, the ApTOLL molecule of the present invention is an aptamer selected from SEQ ID NO: 1-24, particularly selected from SEQ ID NO: 1-16, and more particularly selected from SEQ ID NO: 1-4. In a more particular embodiment, the aptamer is SEQ ID NO: 1.

In other embodiments, the ApTOLL molecule is a variant and/or a derivative of an aptamer selected from SEQ ID NO: 1-24, particularly from SEQ ID NO: 1-16, more particularly of SEQ ID NO: 1-4, and even more particularly of SEQ ID NO: 1. In some embodiments, the ApTOLL molecule is a variant and/or a derivative having at least 70% of sequence identity to SEQ ID NO: 1-24, wherein the variant and/or a derivative is derived from SEQ ID NO: 1-24 and maintains the capability of specifically binding to and reducing and/or inhibiting TLR-4 activation. Particularly, the ApTOLL molecule comprises a sequence at least 85% identical to SEQ ID NO: 1-24, and more particularly a sequence at least 90% or 95% identical to SEQ ID NO: 1-24. In some embodiments, the ApTOLL molecule is a variant and/or a derivative having at least 70% of sequence identity to SEQ ID NO: 1-16, wherein the variant and/or a derivative is derived from SEQ ID NO: 1-16 and maintains the capability of specifically binding to and reducing and/or inhibiting TLR-4 activation. Particularly, the ApTOLL molecule comprises a sequence at least 85% identical to SEQ ID NO: 1-16, and more particularly a sequence at least 90% or 95% identical to SEQ ID NO: 1-16.

In other embodiments, the ApTOLL molecule is a chemically modified aptamer of an aptamer selected from SEQ ID NO: 1-24, particularly SEQ ID NO: 1-16, more particularly of SEQ ID NO: 1-4, and even more particularly of SEQ ID NO: 1. Chemically modifications of the aptamers include base modifications (e.g., 2′-O-methyl U, 2′-O-methyl C), backbone modifications (e.g., 3′-alkylene phosphonates, PNA, T in inverted position), and sugar modifications (e.g., LNA), among other modifications.

In some embodiments, the ApTOLL molecule has a length of between about 40 and about 100 nucleotides. Particularly, the ApTOLL molecule has a length of between about 45 and about 78 nucleotides, and more particularly between about 59 and about 76 nucleotides.

In some embodiments, the ApTOLL molecule has an optional 5′ extension of the nucleotide sequence between 1 and 13 nucleotides in length. In other embodiments, the ApTOLL molecule has an optional 3′ extension of the nucleotide sequence between 1 and 4 nucleotides in length.

In a more particular embodiment:
(a) the ApTOLL molecule has a length between 40 and 100 nucleotides and is selected from the group consisting of SEQ ID NOs: 1, 2, 3, and 4, wherein
(i) the ApTOLL molecule specifically binds to an epitope on the extracellular domain of TLR-4; and,
(ii) binding of the ApTOLL molecule to the epitope reduces and/or inhibits TLR-4 activation; or
(b) the ApTOLL molecule is a functional equivalent variant of the ApTOLL molecule of (a) having at least 85% sequence identity to SEQ ID NO: 1, 2, 3, or 4, wherein the functionally equivalent variant is derived from SEQ ID NO: 1, 2, 3, or 4, and maintains the capability of specifically binding to and reducing and/or inhibiting TLR-4 activation.

In a more particular embodiment, the ApTOLL molecule is SEQ ID NO: 1 (named ApTOLL in APRIL study). In another particular embodiment, the ApTOLL molecule is a variant and/or a derivative of SEQ ID NO: 1 having at least 70% sequence identity to SEQ ID NO: 1 (more particularly 85%), wherein the variant and/or a derivative is derived from SEQ ID NO: 1 and maintains the capability of specifically binding to and reducing and/or inhibiting TLR-4 activation. In another embodiments, the ApTOLL molecule is a chemically modified aptamer of SEQ ID NO: 1.

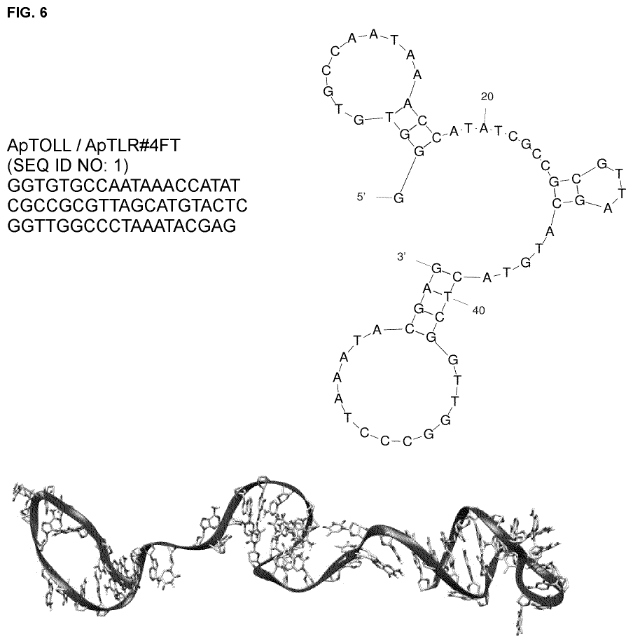

The chemical formula of ApTOLL is C₅₇₅H₇₂₃N₂₂₃O₃₅₁P₅₈ and its molecular weight is 18, 170.80 Da. Tertiary structure of ApTOLL is shown in FIG. 6.

The ApTOLL molecules are formulated in a pharmaceutical composition suitable for administration to a subject. In APRIL study, ApTOLL and placebo were formulated as powder for concentrate for solution for infusion to be reconstituted with 3 ml of water for injections and diluted in a saline bag (100 mL of sodium chloride 9 mg/mL [0.9%] solution for injection) according to the patient weight. The resulting solution can be administered intravenously, e.g., via an infusion pump. In an embodiment, ApTOLL molecules are presented as 1 vial of 7 mg freeze-dried powder for concentrate for solution for infusion for intravenous administration.

In an embodiment, the ApTOLL molecule is formulated in PBS—MgCl₂. In an embodiment, the formulation comprises sodium chloride, potassium chloride, disodium hydrogen phosphate dehydrate, and potassium dihydrogen phosphate to generate a phosphate-buffered solution at pH 7.4, comprising magnesium chloride hexahydrate. This buffer solution and conditions support the aptamer structure and its biological activity. ApTOLL was substituted for A-trehalose dihydrate in the placebo formulations.

### PARTICULAR EMBODIMENTS

As will be apparent to those skilled in the art upon reading this description, each of the individual embodiments described and illustrated herein have discrete components and features which can be combined with the features of any of the other several embodiments without departing from the scope or spirit of the present invention. Particular combinations of the above embodiments detailed in different sections are described herein.

The invention relates to ApTOLL molecules, in particular to an aptamer with SEQ ID NO: 1 (tested in APRIL study) for use in reducing the risk of intracranial hemorrhages after an acute ischemic stroke in a subject, wherein the aptamer is administered after the acute ischemic stroke at a dose between about 0.5 and about 1 mg/kg, and wherein the administration of the aptamer reduces the risk of intracranial hemorrhages between about 5% and about 40% with respect to a subject not treated with the aptamer. Particularly, the administration of the aptamer reduces the risk of intracranial hemorrhages in at least about a 14%. In an embodiment, the intracranial hemorrhages are symptomatic intracranial hemorrhages, and the administration of the aptamer reduces the risk of symptomatic intracranial hemorrhages between about 5% and about 40% with respect to a subject not treated with the aptamer, particularly in at least about a 34%. Particularly, the aptamer is administered at a dose of at least about 0.2 mg/kg.

The invention also relates to ApTOLL molecules, in particular to an aptamer with SEQ ID NO: 1 for use in reducing the neurological damage and in improving the neurological recovery after an acute ischemic stroke in a subject, wherein the reduction of neurological damage and the improvement of neurological recovery is mediated by a reduction of the risk of intracranial hemorrhages, wherein the aptamer is administered after the acute ischemic stroke at a dose between about 0.5 and about 1 mg/kg, and wherein the aptamer:
(i) reduces the infarct volume,
(ii) reduces the NIHSS score, and/or
(iii) increases the mRS score of 0-2 (i.e., reduces the mRS to 0-2),
   with respect to a subject not treated with the aptamer. Particularly, the aptamer is administered at a dose of at least about 0.2 mg/kg.

Particularly, the invention also relates to ApTOLL molecules, in particular to an aptamer with SEQ ID NO: 1 for use in improving the neurological recovery after an acute ischemic stroke in a subject, wherein the improvement of neurological recovery is mediated by a reduction of the risk of intracranial hemorrhages, wherein the aptamer is administered after the acute ischemic stroke at a dose between about 0.5 and about 1 mg/kg, and wherein the aptamer:
(i) reduces the NIHSS score, and/or
(ii) reduces the mRS to 0-2,
   with respect to a subject not treated with the aptamer. Particularly, the aptamer is administered at a dose of at least about 0.2 mg/kg. In another embodiment, the aptamer further reduces the infarct volume.

In an embodiment, the reduction in the infarct volume at 72 hours is between about 10% and about 50% with respect to a subject not treated with the aptamer, particularly of at least about 46%. In another embodiment, the reduction in the infarct volume over time is between about 10% and about 50% with respect to a subject not treated with the aptamer, particularly of at least about 40%.

In an embodiment, the reduction in the NIHSS score at 72 hours is between about 40% and about 60% with respect to a subject not treated with the aptamer, particularly of at least about 57%.

In an embodiment, the increase in the mRS score of 0-2 (i.e., reduction in the mRS to 0-2) at 90 days is between about 10% and about 40% with respect to a subject not treated with the aptamer, particularly of at least about 36%. In another embodiment, the increase in the mRS score of 0-2 (i.e., reduction in the mRS to 0-2) over time is between about 10% and about 50% with respect to a subject not treated with the aptamer, particularly of at least about 31%.

In an embodiment, the aptamer reduces brain edema between about 40% and about 80% with respect to a subject not treated with the aptamer, particularly at least about a 67%.

In an embodiment, the aptamer is administered within about 8 hours from stroke onset, particularly within about 4 hours from stroke onset.

In an embodiment, the aptamer is administered in combination with artery recanalization.

In an embodiment, the aptamer is administered in combination with endovascular treatment. In a particular embodiment, the aptamer is administered prior or concurrently with the endovascular treatment. Particularly, the aptamer is administered less than about 4 hours prior to endovascular treatment.

In an embodiment, the aptamer is administered in combination with pharmacological thrombolysis, particularly the administration of tissue plasminogen activator. Particularly, the aptamer is administered concurrently or after pharmacological thrombolysis.

In an embodiment, the aptamer is administered intravenously by infusion for about 30 minutes.

In an embodiment, the subject:
i) is between about 18 and about 90 years of age;
ii) has a baseline NIHSS of between about 8 points and about 25 points;
iii) has a pre-stroke mRS score of between 0 points and about 2 points;
iv) has an infarct volume of between about 5 cc and about 70 cc;
v) has an occlusion in the TICA, M1 or M2 segments of the middle cerebral artery;
vi) has a mTICI score of 0 or 1;
vii) has a volume of diffusion-weighted imaging restriction between about 5 mL and about 70 mL;
viii) has an ASPECTS between about 6 and about 10; or
ix) any combination thereof,
   at stroke onset.

The contents of all cited references (including literature references, patents, patent applications, and websites) that may be cited throughout this application are hereby expressly incorporated by reference in their entirety for any purpose, as are the references cited therein.

The following examples are offered by way of illustration and not by way of limitation.

### EXAMPLES

### Example 1: APRIL: a Double-Blind, Placebo-Controlled, Randomized, Phase Ib/IIa Clinical Trial of ApTOLL for the Treatment of AIS

### Identification of the Trial: EudraCT: 2020-002059-38 and ClinicalTrials.Gov Identifier: NCT04734548

### Methods and Analysis

### 1.1. Overall Study Design

APRIL is a multicenter, double-blind, randomized, placebo-controlled, parallel groups, Phase Ib/IIa Clinical trial designed to assess the safety & tolerability, pharmacokinetics and biological effect of ApTOLL in AIS patients with confirmed LVO who are candidates to receive endovascular treatment, with or without i.v. tPA. The study population was composed of suitable men and women in which ApTOLL could be administered within 6 h from symptom onset. Allocation in phase IIa was accounted for 3 strata: extent of initial infarct core (<35 vs. ≥35 cc), patient age (<70 vs. ≥70 y) and NIHSS (<15 vs. ≥15). For patients with wake-up stroke, onset time was considered as the time of symptoms first discovered.

The APRIL clinical trial was divided in two parts:
(1) Phase Ib (n=32) a single i.v. administration (30 min infusion), dose escalation with 4 single dose levels (8 patients/level), randomized (1:3); and
(2) Phase IIa (n=119) a single dose, i.v. administration (30 min infusion), parallel (3 arms, placebo: ApTOLL dose A: ApTOLL dose B), randomized (√2:1:1). Therefore, after completion of phase Ib a data safety monitoring board (DSMB) unblinded to study groups selected 2 doses (A, B) to be tested in phase IIa according to initial safety results.

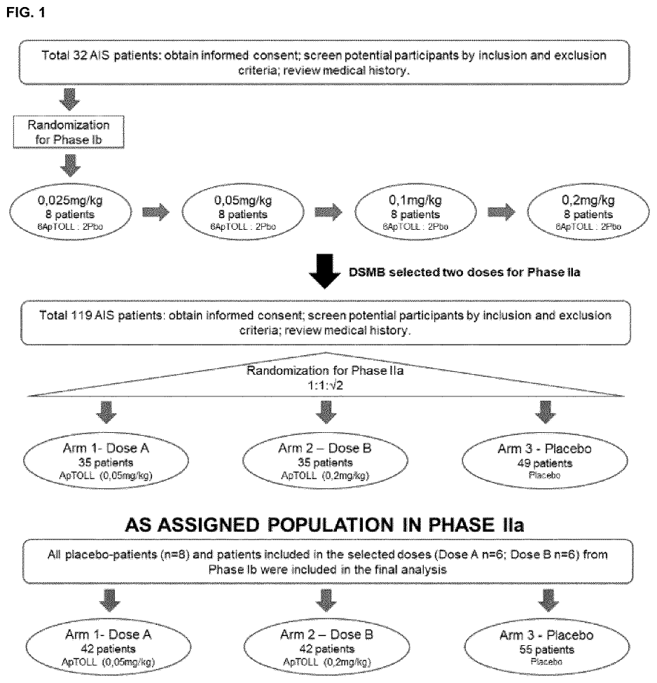

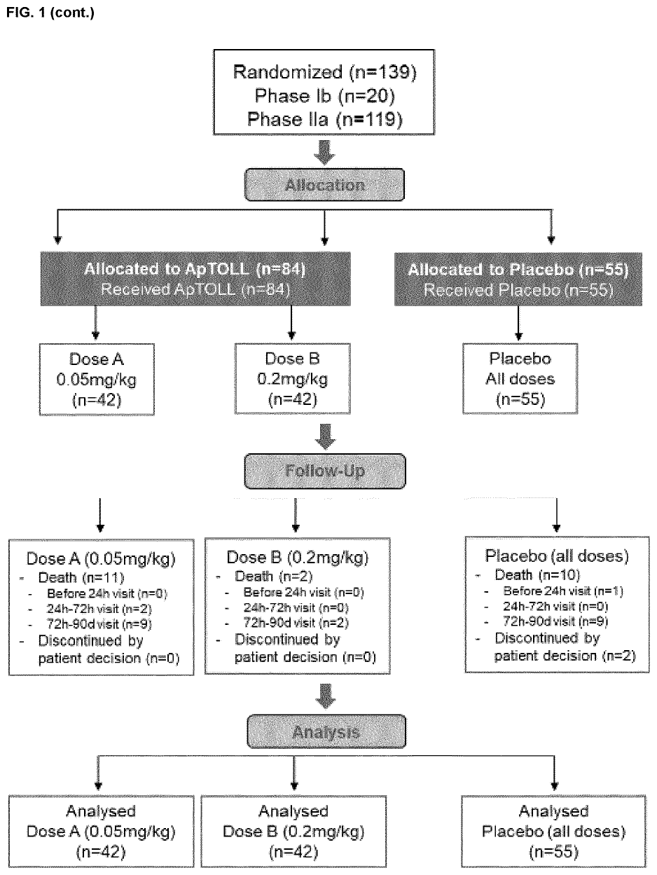

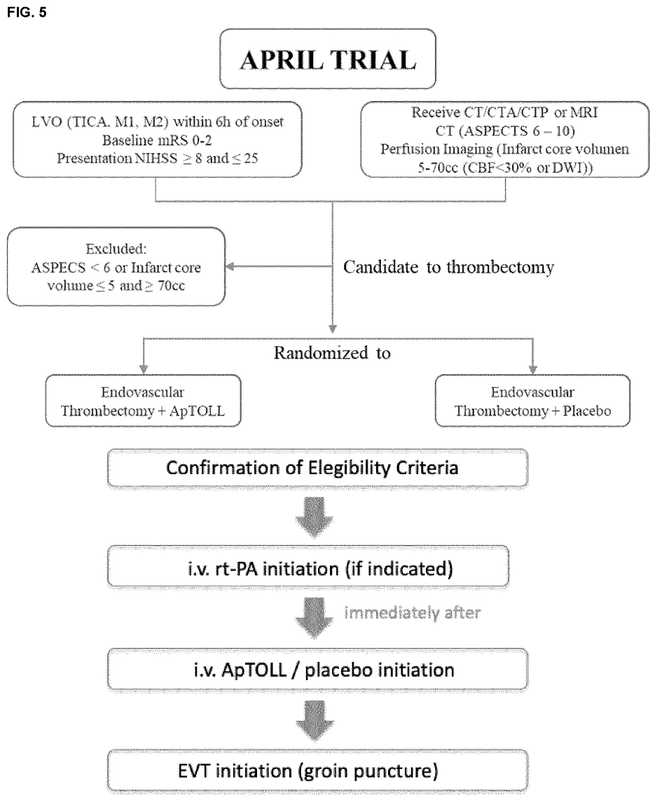

Prior to the enrolment, all patients underwent complete neuroimaging including non-contrast computerized tomography (NCCT), CT angiography and CT perfusion (CTP). Only patients with an indication of EVT, based on NCCT findings (i.e., Alberta Stroke Program Early CT score, ASPECTS>5) were evaluated as candidates for the APRIL study. Further neuroimaging inclusion criteria were the presence of a single LVO at the level of the TICA or the M1 or M2 segments of the middle cerebral artery and the identification of a favorable CTP profile, in order to maximize the chances to identify a biological effect of the study drug. A predicted infarct core volume on CTP defined as CBF<30% between 5 and 70 ml should have to be confirmed with an automated software (RAPID® software) in order to be eligible for the APRIL study. The complete inclusion and exclusion criteria are detailed in section 1.3. After obtaining the informed consent, the patients were randomized to receive EVT+ApTOLL vs. EVT+placebo as shown in FIG. 5. The study medication should have to be administered after imaging acquisition and before EVT initiation (groin puncture). All patients were then treated according to institutional protocols and national and European Stroke Organization (ESO) guidelines. FIG. 1 summarizes the scheme of APRIL study.

### 1.2. Objectives and Endpoints

The aim of the APRIL study was to evaluate if ApTOLL is safe and shows any biological effect in AIS patients with LVO.
Primary objective: to evaluate if the administration of ApTOLL i.v. at ascendant doses is safe and well tolerated as compared to placebo when administered with EVT in the AIS target population.
Secondary objectives: even though it is a unique study, the secondary objectives were different in the two study parts:

### Phase Ib:

To determine the pharmacokinetic profile of ApTOLL in AIS patients, evaluated by the determination of ApTOLL levels in plasma and urine.
To select the two doses to be administered in Phase IIa according to their safety profile.
To provide an initial estimate of the biological effect of ApTOLL on the final infarct volume (measured by MRI-FLAIR [Magnetic Resonance Image-Fluid Attenuated Inversion Recovery] at 72±24 h) and on pro-inflammatory biomarkers linked to AIS (baseline (pre-dose), and at the end of the infusion [up to 1 h], 6 h, 24 h, 48 h and 72 h post-dose).

### Phase IIa:

To assess the biological effect of ApTOLL on the final infarct volume (measured by MRI-FLAIR at 72±24 h) and on pro-inflammatory biomarkers linked to AIS (pre-dose, and 6 h, 24 h, 48 h and 72 h post-dose).
To determine the biological effect of ApTOLL as measured by the functional impairment (NIHSS [National institute of Health Stroke Scale]) at 72 h or discharge (whatever occurs first) and disability (modified Ranking Score [mRS]) at 90 d post stroke.

To achieve these objectives, the following endpoints were evaluated:
Primary endpoints: safety of ApTOLL when combined with EVT as determined by death rate, adverse events that occur during the study, physical examination, laboratory tests, recurrent stroke rate and rate of symptomatic intracranial hemorrhage (sICH).
Secondary endpoints:
   1. Mean final infarct volumes measured at 72±24 h (MRI).
   2. Proinflammatory markers in blood.
   3. Early clinical course (NIHSS at 72 h).
   4. Long-term outcome (mRS at 90 d).
   5. Neuroimage proinflammatory biomarkers in MRI at 72 h and 90 d post-stroke (sub-study conducted only in some APRIL Spanish sites).

### 1.3. Enrollment Inclusion and Exclusion Criteria

Participants were recruited at the participating comprehensive stroke centers in Spain and France. Mechanical thrombectomy includes the use of any commercially available stent retrievers or aspiration catheters or combinations of the above as recommended by ESO guidelines.

### 1.3.1. Inclusion Criteria

1. Age≥18 and ≤90 years.
2. Informed consent obtained from subject or acceptable subject surrogate (i.e., next of kin, or legal representative).
3. A new focal disabling neurologic deficit consistent with acute cerebral ischemia.
4. Baseline NIHSS obtained prior to randomization ≥8 points and ≤25 points.
5. Pre-stroke mRS score of 0-2.
6. Treatable as soon as possible and at least within 6 h of symptom onset, defined as point in time when the subject was last seen well (at baseline). For wake-up strokes, onset time was considered as the time of symptoms first discovered. (Treatment start is defined as study drug administration).
7. Candidates to receive EVT treatment with or without i.v. tPA. For such patients candidates to i.v. tPA therapy, tPA should be initiated as recommended by the European Stroke Organization for the early management of patients with AIS, it means, as soon as possible and within 4.5 h of stroke onset (onset time is defined as the last time when the patient was witnessed to be well at baseline), with investigator verification that the subject had received/was receiving the correct i.v. tPA dose for the estimated weight. Should for any reason i.v. tPA was prematurely halted the cause and the total administered dose was recorded. Additionally, once patient was enrolled in the study, if recanalization was observed and documented before thrombectomy, the patient continued in the trial and no protocol deviation was registered.

### Specific Neuroimaging Inclusion Criteria

8. Occlusion (TICI 0 or TICI 1 flow), of the TICA, M1 or M2 segments of the middle cerebral artery, suitable for mechanical embolectomy, confirmed on CTA. Tandem extra-intracranial lesions may be included.
9. The following imaging criteria should also be met on admission neuroimaging:
a) MRI criterion: volume of DWI (Diffusion-weighted Imaging) restriction≥5 mL and ≤70 mL determined by RAPID® software, OR
b) CT criterion: Alberta Stroke program early CT score (ASPECTS) 6 to 10 on baseline CT AND infarct core determined on admission CTPerfusion by CBF<30%: ≥5 mL and ≤70 mL determined by RAPID® software.
NOTE: ASPECTS were established following the investigator criterium.
10. The subject had an indication and was planned to receive endovascular treatment of stroke according to the ESO Guidelines.

### 1.3.2. Exclusion Criteria

1. Subject had suffered a stroke in the past 1 year.
2. Occlusion (TICI 0 or TICI 1 flow) of the basilar or vertebral or posterior or anterior cerebral arteries.
3. Clinical symptoms suggestive of bilateral stroke or stroke in multiple territories.
4. Known hemorrhagic diathesis, coagulation factor deficiency, or oral anticoagulant therapy with INR>3.0.
5. Baseline platelet count<50,000/μL.
6. Baseline blood glucose of <50 mg/dL or >400 mg/dL.
7. Severe, sustained hypertension (systolic blood pressure>185 mmHg or diastolic blood pressure>110 mmHg).
NOTE: If the blood pressure could be successfully reduced and maintained at an acceptable level using European Stroke Organization (ESO) guidelines recommended medication (including i.v. antihypertensive drips), the patient could be enrolled.
8. Serious, advanced, or terminal illness with anticipated life expectancy of less than 1 year.
9. Subjects with identifiable intracerebral tumors (meningioma was considered extracerebral tumor, so it was not included in this exclusion criterium).
10. History of life-threatening allergy (more than rash) to contrast medium.
11. Known renal insufficiency with creatinine≥3 mg/dL or Glomerular Filtration Rate (GFR)<30 mL/min.
12. Cerebral vasculitis.
13. Evidence of active systemic infection.
14. Known current use of cocaine at time of treatment.
15. Patient participating in a study involving an investigational drug or device that would impact this study.
16. Patients that were unlikely to be available for a 90-day follow-up (e.g., no fixed home address, visitor from overseas).
17. Female who was pregnant or lactating or had a positive pregnancy test at time of admission.

### Specific Neuroimaging Exclusion Criteria

18. CT or MRI evidence of hemorrhage (the presence of microbleeds was allowed).
19. Significant mass effect with midline shift.
20. Suspicion of aortic dissection presumed septic embolus, or suspicion of bacterial endocarditis.

### 1.4. Enrollment and Randomization

All patients who met the eligibility criteria were eligible for APRIL clinical trial enrollment. This included both, patients directly admitted to the study site and patients transferred from a primary stroke center (drip and ship).

### 1. Treatment Blinding

The study was masked in a double-blind fashion, i.e., neither patient nor treating physicians were aware of the administered treatment. The solution of ApTOLL or matching placebo was limpid, transparent, and colorless.

### 2. Enrollment and Randomization Phase Ib

During the Phase Ib, four ascending dose levels (0.025, 0.05, 0.1 and 0.2 mg/kg) were completed. Within each dose level group, 8 patients were randomized to receive ApTOLL plus EVT vs. Placebo plus EVT at 3:1 ratio.

A staggered dosing scheme was used to maximize the safety of the patients. After each dose level (follow up 72 h visit of last patient included in the corresponding dose level), the DSMB evaluated the safety results and approved the next dose level, if appropriate. The safety parameters to be evaluated were (1) any SUSAR (suspected serious adverse reactions), SAE (serious adverse event) or AE that could be related to the drug administration and (2) blood biochemistry parameters.

3. Transition from Phase Ib to Phase IIa

Before starting the Phase IIa, and once safety data from the last patient in Phase Ib was obtained, the DSMB evaluated the whole safety results to select the two optimal doses to be studied in Phase IIa (Dose A, Dose B):

### 1. Safety Parameters Related to Stroke:

Death.
Intracranial hemorrhage (ICH) and hemorrhagic transformation (HT) resulting in new symptoms or in worsening of the existing ones.
Brain edema resulting in herniation and neurological worsening or death. Further potential AEs related to stroke: Epileptic seizures; cardiac conduction disturbances, arrythmias; effects on coagulation and fibrinolysis; hypotension/hypertension; hyperglycemia; hyperthermia; severe infections; deep vein thrombosis, pulmonary embolism and venous thromboembolism; vomiting; and anxiety, hallucinations, agitation.

### 2. Safety Parameters Related to the Drug:

Complement activation: CH50 (50% hemolytic complement) and C3/C4 (Complement factors 3 and 4) levels.
Biochemistry: CK (Creatine Kinase) and CRP (C-Reactive Protein)
Coagulation parameters: aPTT (activated Partial Thromboplastin Time), Prothrombin activity (PT) and INR (International Normalized Ratio).

### 4. Enrollment and Randomization in Phase IIa

In Phase IIa, three arms were studied (placebo, and ApTOLL at dose A or B). Eligible patients were randomized into placebo plus EVT vs. ApTOLL (dose A) plus EVT vs. ApTOLL (dose B) plus EVT, at √2:1:1 ratio which, in turn, yielded a probability of random assignment of 0.41, 0.29 and 0.29, respectively.

In Phase IIa, the DSMB analyzed AEs and SAEs when approximately 20 patients had been included in each treatment group and all of them have reached the 72 h follow-up visit. In both Phase Ib and Phase IIa, the CAC (Clinical Adjudication Committee) analyzed all AEs and SAEs to determine which ones were related to the medication and communicated this decision to the DSMB. With this information, DSMB reviewed the reported AEs and SAEs.

### 1.5. Study Treatments

Both treatments (ApTOLL and placebo) were administered intravenously using an infusion pump during 30 min.

### 1. Study Treatment

ApTOLL was presented as 1 vial of 7 mg powder for concentrate for solution for infusion for i.v. administration. ApTOLL and placebo were formulated as powder for concentrate for solution for infusion to be reconstituted with 3 ml of water for injections and diluted in a saline bag (100 ml of sodium chloride 9 mg/mL [0.9%] solution for injection) according to the patient weight. The placebo treatment matched the appearance of ApTOLL when as powder for concentrate for solution for infusion and when reconstituted. The formulation with ApTOLL consisted of a lyophilized containing sodium chloride, potassium chloride, disodium hydrogen phosphate dehydrate, and potassium dihydrogen phosphate to generate a phosphate-buffered solution at pH 7.4, comprising magnesium chloride hexahydrate. API (i.e., ApTOLL) was substituted for A-trehalose dihydrate in the placebo formulations.

Example for a 70 kg patient in level 3, 7 mg should be administered, so 3 mL from the stock solution should be taken and subsequently diluted in 100 ml of sodium chloride 9 mg/ml (0.9%) solution for injection.

The administration of ApTOLL/placebo should be started before EVT is initiated (before groin puncture). The rate of infusion should not exceed 2.67 mg/min (equivalent to 3.33 mL/min when reconstituted and diluted) at every case. Before reconstitution and dilution, ApTOLL and placebo were stored at −20° C. The reconstitution was done just before the administration (maximum 10 min before injecting in the saline bag).

The dose scheme for Phase Ib was as follows (Table 1):

TABLE 1
Dose escalation in Phase Ib.

| | | Number of |
|---|---|---|
| Dose | Dose of | patients enrolled |
| level | ApTOLL (mg/kg) | (Ratio ApTOLL:placebo) |
| 1 | 0.025 | 8 (6 ApTOLL:2 placebo) |
| 2 | 0.05 | 8 (6 ApTOLL:2 placebo) |
| 3 | 0.1 | 8 (6 ApTOLL:2 placebo) |
| 4 | 0.2 | 8 (6 ApTOLL:2 placebo) |

### 2. Medications/Treatment Permitted and not Permitted Before and During the Clinical Study

In addition to the study drug, patients were treated according to the ESO guidelines.

### 1.6. Study Assessments and Procedures

The flow of treatment is the displayed in FIG. 5. Each subject underwent the following procedures (Table 2). The study duration for each subject was a maximum of 90 days.

TABLE 2
Schedule of Events

| | Baseline | | | | | Follow |
|---|---|---|---|---|---|---|
| | information - | | Follow | Follow | Follow | up |
| | Study | Throm- | up | up | up | 90 |
| Assessments | Allocation | bectomy | 24 h | 48 h | 72 h | days |
| Admission Details | X | | | | | |
| Demographics and | X | | | | | |
| physical examination | | | | | | |
| Medical History | X | | | | | |
| and baseline medication | | | | | | |
| Eligibility Criteria | X | | | | | |
| Informed Consent | X | | | | | |
| Vital sings (heart rate | X | | X | X | X | |
| (HR), blood pressure | | | | | | |
| (BP) and body | | | | | | |
| temperature (BT)) | | | | | | |
| mRS | X | | | | X | X |
| NIHSS | X | | X | X | 2X¹ | X |
| Pregnancy test | X | | | | | |
| Randomization | X | | | | | |
| Blinded Study | X | | | | | |
| medication administration | | | | | | |
| Monitorization of study | X | X | X | X | X | |
| medication site of injection | | | | | | |
| Blood test: Hematology, | X | | X | | | |
| Coagulation (including | | | | | | |
| INR) and Biochemistry | | | | | | |
| Blood test: aPTT, PCR | X | | X | X | X | |
| and complement activation | | | | | | |
| Proinflammatory biomarkers | 3X² | | X² | X² | X² | |
| in blood (Phase Ib) | | | | | | |
| Proinflammatory | 2X² | | X² | X² | X² | |
| biomarkers (Phase IIa) | | | | | | |
| Pharmacokinetics | 3X³ | | X³ | X³ | X³ | |
| (in Phase Ib) | | | | | | |
| Urine analysis for | | X⁴ | X⁴ | | | |
| elimination assessment | | | | | | |
| (in Phase Ib) | | | | | | |
| ECG | X⁵ | X | X | X | | |
| Non-Contrast CT | X | | X | | | |
| CTA | X | | | | | |
| CT-P | X | | | | | |
| FLAIR-MRI | X⁶ | | | | X | X⁷ |
| EVT Details⁸ | | X | | | | |
| Angiogram | | X | | | | |
| Stroke etiology | | | | | | X⁹ |
| EuroQol EQ-5D | | | | | | X |
| Relevant Meds | X | X | X | X | X | X |
| (patient's medical history) | | | | | | |
| Adverse Events | X | X | X | X | X | X |

X¹- NIHSS performed at day 5 or discharge (whatever occurs first).
X²- In Phase Ib, proinflammatory biomarker samples taken at pre-dose, and at the end of the infusion (up to 1 h), 6 h, 24 h, 48 h and 72 h post-dose. In Phase IIa, proinflammatory biomarkers were analyzed at pre-dose and 6 h, 24 h, 48 h and 72 h post-dose.
X³- Pharmacokinetic samples taken at pre-dose, and at the end of infusion (up to 1 h), 6 h, 24 h, 48 h and 72 h from the end of infusion.
X⁴- Collect, if possible, urine samples from study drug administration to 24 h.
X⁵- First ECG performed at 6 h post-dose.
X⁶- Included only in those hospitals where the inclusion was performed based on MRI criteria (i.e., France).
X⁷- Only for sites participating in image sub-study.
EVT Details⁸- Groin puncture done before 8 h after symptoms onset. For wake-up strokes, onset time was considered as the time of symptoms first discovered.
X⁹- Stroke etiology was completed when available.

Endovascular thrombectomy (EVT details): EVT was initiated (groin puncture) after randomization and administration of the study drug. EVT was initiated within 8 hours from symptoms onset (ApTOLL was administered within 6 h after stroke onset) (for wake-up strokes, onset time was considered as the time of symptoms first discovered). Individual investigators used any approved device or any combination of devices to remove thrombus from the TICA, MCA M1 segment or, if needed, from M2 segments of the intracranial circulation.

### Imaging Procedures: Non-Contrast CT, CTA, CTP and MRI.

CT images were read by appropriately trained local clinicians. ASPECTS for patient selection were determined independently by the local clinicians. CTP images with mismatch determination were read by iSchemaView automated RAPID software. All brain imaging from stroke onset through hospital discharge, including the MRI, CTP/MRP, and CT, as well as angiographic images obtained for the diagnostic and therapeutic portions of the procedure, were assessed independent of each other and blinded to treatment allocation at the APRIL Central Imaging Core Lab (ICL). The following imaging and angiography variables were extracted based on direct prior experience with these measures and scales, using the published definitions and standards:

### Baseline:

ASPECTS
Ischemic core-post hoc DWI or CTP rCBF<30% volumes when available
Tmax>6 sec volume (if CTP or PWI available)
Hemorrhage
Baseline occlusive lesion location
CTA collateral score-based on availability (post hoc)

### Procedure:

Presence of stenosis proximal to arterial occlusive lesion
Arterial occlusive lesion
Collateral flow grade-ASITN
eTICI on each device pass
Distal emboli
Emboli to new territories
Dissection
Vessel perforation

### Post-Procedure (24 Hour):

Infarct volume
Hemorrhage (Heidelberg)

### Post-Procedure (72 Hour):

Final infarct volume post hoc DWI (or on CT if MRI is not available)·
Hemorrhage (Heidelberg)

### Neurological Deterioration or Course Post-Procedure:

Relevant imaging findings and measures, based on availability (infarct volume for all available follow-up images)

In Phase IIa, a second MRI was performed at day 90 post-procedure in the context of the image sub-study and total and relative (grey/white matter) sizes of infarct volume at 90 d were determined. In addition, there were measured brain perfusion parameters from IVIM (Intravoxel Incoherent Motion) sequence at 72±24 h, microstructural indexes from DTI (Diffusor Tensor Imaging) at 72±24 h and 90 d, and iron load in brain tissue at 72±24 h and 90 d.

Fibrinolytic therapy: Patients received standard ESO guidelines directed medical therapy, which could include i.v. tPA infusion in patients presenting within the first 4.5 hours from last-seen-normal and meeting other ESO label criteria. Post-tPA patients were treated based on standard study site protocols for these patients. Time of tPA infusion and dose administered was collected.

### Results

### 1.7. Study Design Performed and Participants

APRIL is a double-blind, randomized, multicenter, placebo-controlled, Phase Ib/IIa Clinical Study, designed to assess if the administration of ApTOLL in stroke patients with confirmed LVO who are candidates to receive EVT with or without i.v. thrombolysis is safe, well tolerated and shows any biological effect. The trial was done at 15 comprehensive stroke centers in Spain (12 sites) and France (3 sites). The study was approved by the ethics committee at each site and by the national regulatory authorities. Signed informed consent was obtained from the patients or their legally authorized representative.

Eligible patients were men and non-pregnant women aged 18 to 90 years old with a disabling ischemic stroke at the time of randomization (baseline National Institutes of Health Stroke Scale [NIHSS] 5 to 25; range 0-42, with higher scores indicating greater stroke severity); functionally independent before the stroke, defined as a modified Rankin Scale (mRS) score between 0 and 2, with scores ranging from 0 (no symptoms) to 6 (death); in which ApTOLL could be administered within 6 h from symptom onset. For patients with a wake-up stroke, onset time was considered as the time of symptoms first discovered, as safety was not expected to be challenged if the study drug was administered beyond 6 hours from actual stroke onset.

Patients with a single LVO at the level of the TICA or the M1 or M2 segments of the middle cerebral artery on non-invasive vascular imaging that were considered to undergo EVT, based on noncontrast Computed Tomography (NNCT) findings (Alberta Stroke Program Early CT Score [ASPECTS] of 6-10; range 0-10, with 1 point subtracted for any evidence of early ischemic change in each defined region on the CT scan), were evaluated as candidates for the APRIL study. In order to maximize the chances to identify a biological effect of the study drug, the identification of a specific CT perfusion (CTP) profile as previously defined (Olivé-Gadea M et al., 2021) was also required for enrollment. A predicted infarct core volume on CTP defined as relative Cerebral Blood Flow<30% between 5 and 70 ml had to be identified with a previously validated automated software (RAPID®, iSchemaView) in order to be eligible for the APRIL study.

Qualifying imaging was planned to be acquired at the endovascular center; in a minority of cases (n=2) qualifying imaging was done at a primary stroke center before transfer to the thrombectomy center. No screening log was kept. A full list of eligibility criteria is available in section 1.3 of EXAMPLE 1.

### 1.8. Enrollment, Randomization and Masking

The APRIL clinical trial was divided into two parts. In Phase Ib 32 patients were allocated (6 ApTOLL: 2 placebo each level) in four ascending doses (0.025, 0.05, 0.1 and 0.2 mg/kg over a 30 minutes i.v. infusion). A staggered dosing regime was used to maximize patient safety. After the results of the 72 hours follow-up visit were evaluated by the Data Safety Monitoring Board (DSMB), the next dose level was approved, if deemed appropriate. The safety outcomes evaluated were any suspected serious adverse reaction, serious adverse event (SAE) or adverse event (AE) that could be related to the drug administration and blood biochemistry parameters.

In both Phase Ib and Phase IIa, the masked Clinical Adjudication Committee analyzed all AEs and SAEs to determine their potential relation to the study medication. With this information, the masked DSMB reviewed all reported AEs and SAEs. After completion of Phase Ib, the DSMB was instructed to select the two doses to be tested in the following Phase IIa according to initial safety results. Patients treated with either placebo or one of the two selected doses in Phase Ib were further analyzed together with patients enrolled into Phase IIa. In Phase IIa, three arms were studied and patients were randomized into one of the two selected doses of ApTOLL or placebo in a 1:1:√2 ratio which, in turn, yields probabilities of assignment of 0.293, 0.293 and 0.414, respectively. ApTOLL and placebo were prepared as colorless solutions in numbered, refrigerated (−20° C.) vials. These vials were visually identical, except for a unique vial number, so that all trial personnel and patients were fully masked to treatment allocation.

In both study phases, patients were randomly assigned by using a real-time, internet-based system. This process was automated from study startup and allowed for complete concealment of the sequence of allocation. The randomization system was originally planned to execute stratified allocation according to three strata: extent of predicted infarct core (<35 vs. ≥35 cc) on admission CTP, patient age (<70 vs. ≥70 y) and admission NIHSS (<15 vs. ≥15). However, due to an error in the system that went unnoticed until study termination, stratification was not applied. To account for the potential bias introduced by this error, post-hoc analyses of the primary outcome adjusting for the preplanned stratifying factors were reported.

During Phase IIa, after 100 patients completed the 72 hours follow-up, the DSMB performed a preplanned interim analysis and decided not to adopt any changes in the protocol for the remaining patients. FIG. 1 summarizes the scheme of APRIL study.

### 1.9. Procedures

After qualifying imaging, all patients received EVT, and i.v. thrombolysis if indicated (before or during EVT, at a primary hospital before transfer or at the endovascular center) and were treated according to local institutional protocols and national and European Stroke Organization guidelines (Turc G et al., 2019). Patients had to meet inclusion and exclusion criteria at the thrombectomy-capable center. The trial drug was administered as soon as possible after randomization, and investigators were asked to ensure that the drug infusion was started after imaging acquisition and before initiation of the EVT (arterial puncture).

Sites were expected to adhere to national guidelines for stroke unit, stroke rehabilitation, and stroke prevention care. All patients had standard assessments of demographic characteristics, past medical history, laboratory values, and stroke severity (NIHSS score). The quality of reperfusion after EVT was assessed with the expanded Thrombolysis in Cerebral Ischemia (eTICI) scale. Follow-up brain imaging was performed at 24 h with CT and at 72 hours with MRI, if possible. Clinical follow-up was obtained at 90 days from randomization, if possible in-person, to assess the degree of disability as determined by the mRS. Where in-person follow up was not possible, video conferencing or telephone follow-up was obtained. In all patients taking in Phase Ib, up to 6 consecutive blood samples (baseline, 1, 6, 24, 48 and 72 hours after randomization) were drawn before and after dosing for pharmacokinetic analysis of ApTOLL concentrations and determination of plasma concentration of different pro-inflammatory biomarkers linked to acute stroke response. In Phase IIa, blood biomarkers were determined at 5 timepoints (baseline and 6, 24, 48 and 72 hours after randomization). Imaging interpretation was blinded and performed at a central core laboratory (UCLA, Los Angeles, Ca). Clinical data was verified by independent monitors (Anagram-ESIC, Barcelona, Spain).

### 1.10. Outcomes

The primary objective of the study was to evaluate if the administration of ApTOLL intravenously at different doses was safe and well tolerated, as compared to placebo, when administered with EVT, and i.v. fibrinolysis when indicated. The safety of ApTOLL was determined by monitoring adverse events that occurred during the study, identified on physical examination, by laboratory tests or by neuroimaging. The primary endpoint was defined as the presence of any of the following events: death of any cause, intracranial hemorrhage resulting in new symptoms or in worsening of the existing ones (sICH, symptomatic intracranial hemorrhage), brain edema resulting in herniation, neurological worsening or death, and recurrent stroke.

Even though APRIL was a unique study, the secondary objectives were different in the two study parts:
Phase Ib: (1) To determine the pharmacokinetic profile of ApTOLL in AIS patients, evaluated by the determination of ApTOLL levels in plasma and urine. (2) To select the two doses to be administered in Phase IIa according to their respective safety profiles. (3) To provide an initial estimate of the therapeutic effect of ApTOLL on the final infarct volume (measured by MRI-Fluid Attenuated Inversion Recovery [MRI-FLAIR] at 72±24 h) and on pro-inflammatory biomarkers (baseline [predose], and at the end of the infusion [up to 1 h], 6 h, 24 h, 48 h and 72 h post-dose).
Phase IIa: To assess the therapeutic and clinical effect of ApTOLL on: (1) The final infarct volume (measured by MRI-FLAIR at 72±24 h); (2) pro-inflammatory plasma biomarkers (pre-dose, and at 6 h, 24 h, 48 h and 72 h post-dose), (3) early clinical course (NIHSS at 72 h or discharge [whatever occurs first]), and (4) long term functional outcome (mRS at 90 days post stroke).

In those cases where one or more NIHSS measurements were missing, the last measure post study drug administration was considered. Deceased patients were included in the “as assigned population” with an mRS score of 6. For patients known to be alive at 3 months post randomization in whom follow-up evaluations were not possible, the discharge mRS was carried forward. In cases where the MRI at 72 hours could not be obtained, the final infarct volume was determined in the last CT available after ApTOLL administration. If no follow-up image was available, final infarct volume was considered as missing.

### 1.11. Statistical Analysis

A statistical analysis plan (SAP) including the list of all tables, listings and graphs were issued before the study database lock.

Statistical analyses were performed by an independent external statistical consulting group. All analyses were performed on the as assigned population, defined as all patients randomly allocated in the trial, regardless of treatment received. The total sample size was defined to be 151 patients: Phase Ib=32 patients and Phase IIa=119 patients. Due to the exploratory nature of study, there was no estimate of the statistical power. The analyses assumed a progressive relationship (i.e., any dose above an unsafe dose was considered also unsafe). Patients enrolled in the Phase Ib who received either placebo or one of the two selected doses of ApTOLL were combined with the Phase IIa patients. To successfully combine the patients from both study phases, the design, the follow-up and data collection were strictly identical in Phase Ib and Phase IIa parts of the trial, with the only exception of sequential blood and urine sampling for pharmacokinetic analyses purposes that were performed only in Phase Ib.

Categorical variables were summarized by means of counts and percentages. Continuous variables were summarized by the mean, the standard deviation (SD), the median and the corresponding inter-quartile range. Where informative, these summaries were reported by treatment and visit. Infarct volumes showed a skewed distribution and were reported as median (IQR).

The primary outcome of the study was compared between groups based on the absolute difference in proportions and asymptotic Wald confidence intervals at 95%. When both proportions to compare were zero, a generalized linear model was employed. For the confidence interval of the odds ratio, asymptotic Wald confidence limits at 95% based on a log transformation of the odds ratio was employed. Between groups, differences in continuous secondary outcomes (final infarct volume and NIHSS score at 72 hours) were tested in a one-way analysis of variance after adjustment by the Scheffe method; mean difference and 95% confidence intervals between placebo and ApTOLL doses are reported. To evaluate the shift analysis on the mRS score at 90 days, a proportional odds ordinal logistic regression model was built, with placebo as the reference category, to estimate the unadjusted common odds ratio and 95% confidence intervals for a better outcome at 90 days.

In order to account for a potential imbalance in baseline patient characteristics due to the lack of stratified randomization, a post-hoc binomial logistic regression model was performed adjusted for predicted infarct core volume on admission, age and baseline NIHSS score to estimate the odds ratio and 95% confidence intervals. A fully specified Statistical Analysis Plan was reported before completing the study. The trial is registered as EudraCT: 2020-002059-38 and ClinicalTrials.gov: NCT04734548.

1.12. Results. Study Population

Between November 2020, and June 2021, 32 patients were enrolled in Phase Ib, and randomly assigned to receive ApTOLL or placebo (FIG. 1) to complete the four dose escalation groups. ApTOLL plasma concentrations were obtained from 22 patients in Phase Ib. The DSMB did not find any safety concern and selected the 0.05 mg/kg and 0.2 mg/kg doses to be used in Phase IIa. The 0.05 mg/kg dose was selected because it should be sufficient to produce some benefit and all patients achieved sufficient concentrations of ApTOLL in the pharmacokinetic analysis. The 0.2 mg/kg dose was expected to achieve the maximum therapeutic benefit since this dose reached the maximum bioavailability in the first-in-human study Hernandez-Jimenez M et al., 2022. Additionally, there did not appear to be any toxicity risks limiting this choice.

From July 2021 to April 2022, 119 patients were assigned to receive either ApTOLL 0.05 mg/kg (n=36), ApTOLL 0.2 mg/kg (n=36) or placebo (n=47). The pooled Ib/IIa population was composed of 42 patients allocated to ApTOLL 0.05 mg/kg, 42 patients allocated to ApTOLL 0.2 mg/kg and 55 patients allocated to placebo (FIG. 1). Baseline characteristics were similar between groups (Table 3). Patient's compliance with eligibility criteria and treatment administration, major protocol noncompliances, patient's withdrawals and the reason of withdrawal (e.g., AE, protocol noncompliance, lost to follow-up, consent withdrawal and other reasons) and assignment to each analysis population were reported. Major protocol deviations occurred in 10 patients.

Of the 151 patients in the pooled Ib/IIa population, all received the allocated intervention (either active dose or placebo) and no crossovers occurred. All patients received the assigned intervention although 3 had incorrect volume or duration: 2 (4.8%) with ApTOLL 0.05 mg/kg and 1 (1.82%) with placebo. All patients except one in ApTOLL 0.2 mg/kg group (99.3%) underwent an attempted EVT; i.v. thrombolysis was administered in 29 (69.1%) patients in the ApTOLL 0.05 mg/kg group, 27 (64.3%) patients in the ApTOLL 0.2 mg/kg group and 29 (52.7%) patients in the placebo group. The overall workflow (onset to randomization, onset to study drug administration, and study drug to reperfusion) and quality of reperfusion (on the expanded eTICI scale) were similar in all groups (Table 3).

1.13. Results. Outcomes

At 90 days, primary safety outcome data were missing for 2 patients (1.3%; 2 patients withdrew consent) (FIG. 1). Death of any cause occurred in 10 patients allocated to placebo (18.2%), in 11 patients allocated to ApTOLL 0.05 mg/kg (26.2%; absolute difference vs. placebo 8%; 95% CI: −9% to 25%), and in 2 patients allocated to ApTOLL 0.2 mg/kg: 3 (4.8%; absolute difference vs. placebo −13%; 95% CI: −25% to −1%). Table 4 shows the distribution of primary and secondary endpoints in the study groups.

The final infarct volume was 44 mL (IQR 26-89) in patients allocated to placebo, 46 mL (IQR 18-100) in patients allocated to ApTOLL 0.05 mg/kg (mean difference of log-transformed final infarct volume vs. placebo −12%; 95% CI: −49% to 35%) and 23.5 mL (IQR 13-42) in patients allocated to ApTOLL 0.2 mg/kg (mean difference of log-transformed final infarct volume vs. placebo −42%; 95% CI: −66% to 1%). The NIHSS assessed at 72 hours was 7 (IQR 3-17) in patients allocated to placebo, 8 (IQR 3-17) in patients allocated to ApTOLL 0.05 mg/kg (mean difference of log-transformed 72 hours NIHSS vs. placebo −1%; 95% CI: −41% to 40%) and 3 (0-11) in patients allocated to ApTOLL 0.2 mg/kg (mean difference of log-transformed 72 hours NIHSS vs. placebo −45%; 95% CI: 67% to −10%). The rate of patients with mRs score 0-2 at 90 days was 47.1% in patients allocated to placebo, 37.5% in patients allocated to ApTOLL 0.05 mg/kg (common odds ratio for a better outcome vs. placebo 0.76 95% CI 0.37-1.56) and 64.3% in patients allocated to ApTOLL 0.2 mg/kg (common odds ratio for a better outcome vs. placebo 2.44, 95% CI 1.76-5.00). The analyses of the multiple blood biomarkers were not significantly different among the studied groups. FIGS. 2-4 show a graphical representation of secondary efficacy outcomes.

### 1.14. Discussion

The APRIL study showed that the infusion of ApTOLL 0.2 mg/kg (SEQ ID NO: 1) in combination with EVT in selected stroke patients is safe and reduces the mortality at 90 days. Moreover, the efficacy of ApTOLL as a neuroprotectant drug in acute cerebral ischemia is supported by consistent positive results in most predefined secondary outcome measures including final infarct volume, early neurological impairment and long term disability.

ApTOLL has demonstrated that: (1) effectively reach the tissue at risk exerting a protective effect directly in the area of ischemic penumbra potentially extending the therapeutic window of reperfusion therapies; and (2) limit the phenomena of hemorrhagic transformation and reperfusion damage that may occur after recanalization.

A key strength of the trial is that the design largely matched the preclinical ischemia/reperfusion models of transient middle-cerebral artery occlusion. Patients had a median duration of stroke onset to study drug administration of 210 minutes and had high reperfusion rates after EVT (final eTICI 2b-3 score 87%). The observed pharmacokinetic curves suggest that ApTOLL could be exerting its neuroprotective effect up to 12 hours from administration including the initial period of ischemia and the following hours after reperfusion. The median time from drug infusion to recanalization (when achieved) was approximately 180 minutes, corresponding to the potential neuroprotective time during ischemia.

The subgroup analysis suggests a similar treatment effect in patients who received the drug in the early time window (<3 hours from symptom onset) and those who were treated in the later window (3-6 hours).

The study was not powered to achieve conclusions regarding the efficacy of ApTOLL in improving outcomes in acute stroke patients but to identify a safe dose in these patients. Nevertheless, promising results have been observed in the present phase. More results are shown on Table 5 and FIG. 4.

### 1.15. Conclusions

In AIS, 0.2 mg/kg of ApTOLL administered within 6 h of onset, in combination with EVT, was safe and associated with meaningful clinical effects reducing mortality and disability at 90 days as compared to placebo.

TABLE 3
Baseline characteristics expressed as mean (standard deviation) or median [interquartile range]
(mRS: modified Rankin Scale, NIHSS: National Institutes of Health Stroke Scale, LTSW: last time seen
well, ASPECTS: Alberta Stroke Program Early CT score, ICA: Internal Carotid Artery, MCA: middle cerebral
artery, EVT: endovascular treatment, eTICI: expanded Thrombolysis in Cerebral Ischemia).

| | ApTOLL | ApTOLL | |
|---|---|---|---|
| | 0.05 mg/kg | 0.2 mg/kg | Placebo |
| | (N = 42) | (N = 42) | (N = 55) |
| Age, mean (SD) | 71.29 (10.97) | 68.33 (12.51) | 71.13 (12.86) |
| Female, n (%) | 18 (42.86%) | 18 (42.86%) | 22 (40.00%) |
| Pre-stroke mRS score, | 0 | 0 | 0 |
| median[IQR) | [0-1] | [0-0] | [0-1] |
| Hypertension, n (%) | 29 (69.05%) | 30 (71.43%) | 36 (65.45%) |
| Hyperlipidemia, n (%) | 23 (54.76%) | 23 (54.76%) | 25 (45.45%) |
| Diabetes mellitus, n (%) | 10 (23.81%) | 10 (23.81%) | 9 (16.36%) |
| Atrial fibrillation, n (%) | 11 (26.19%) | 14 (33.33%) | 16 (29.09%) |
| Ischemic Heart disease, n (%) | 4 (9.52%) | 8 (19.05%) | 7 (12.73%) |
| Peripheral vascular disease, n (%) | 3 (7.14%) | 1 (2.38%) | 2 (3.64%) |
| Previous Stroke, n (%) | 5 (11.90%) | 2 (4.76%) | 5 (9.09%) |
| Active Smoker, n (%) | 7 (16.67%) | 9 (21.43%) | 7 (12.73%) |
| Systolic Blood Pressure, | 150.00 (19.82) | 143.98 (19.57) | 148.60 (19.46) |
| mmHG, mean (SD) | | | |
| Diastolic Blood Pressure, | 79.76 (13.03) | 80.45 (11.84) | 79.64 (14.38) |
| (mmHG, mean (SD) | | | |
| Admission NIHSS, median | 19 | 17 | 18 |
| [IQR] | [17-21] | [12-21] | [13-20] |
| Intravenous thrombolysis, n (%) | 29 (69.05%) | 27 (64.29%) | 29 (52.73%) |
| LTSW/onset to fibrinolysis, | 2.03 | 2.07 | 1.92 |
| hours, mean (SD) | [1.53, 2.75] | [1.78, 2.25] | [1.17, 2.15] |
| LTSW/onset to study randomization, | 2.82 | 2.46 | 2.9 |
| hours, mean (SD) | [2.02, 4.05] | [2.04, 3.64] | [2.28, 4.05] |
| LTSW/onset to study drug | 3.28 | 2.78 | 3.37 |
| administration, hours, mean (SD) | [2.75-4.25] | [2.27, 3.8] | [2.55, 4.67] |
| CT-Perfusion on admission | 41 (97.62%) | 41 (97.62%) | 55 (100.0%) |
| (Vs MRI), n (%) | | | |
| Predicted Infarct Core Volume on | 23.22 (17.71) | 20.25 (15.90) | 25.47 (17.40) |
| admission, mL, mean (SD) | | | |
| Median [IQR] | 16 [10-32] | 15 (7.5-26.5] | 23 [9-41] |
| Tmax > 6 seconds on admission | 131.85 (50.00) | 114.00 (60.30) | 142.27 (53.75) |
| perfusion imaging mL, mean (SD) | | | |
| Median [IQR]) | 138 | 105 | |
| | [86-169] | [62-166] | |
| ASPECTS, median [IQR] | 8 | 9 | 8 |
| | [7, 9] | [8, 10] | [7, 8] |

Occlusion location on admission imaging

| | | | |
|---|---|---|---|
| Extra-cranial ICA n (%) | 8 (19.05%) | 3 (7.32%) | 12 (21.82%) |
| TICA n (%) | 7 (16.67%) | 1 (2.44%) | 7 (12.73%) |
| M1-MCA n (%) | 27 (64.29%) | 27 (65.85%) | 42 (76.36%) |
| M2-MCA n (%) | 8 (19.05%) | 15 (36.59%) | 10 (18.18%) |
| Other | 3 (7.14%) | 3 (7.32%) | 2 (3.64%) |
| Time from LTSW to arterial puncture, | 205 (147-277) | 189 (144-277) | 230 (172-324) |
| median (IQR) | | | |
| Duration of EVT procedure, median (IQR) | 52 (29-79) | 35 (25-55) | 47 (29-73) |
| General anesthesia n (%) | 3 (7.32%) | 8 (19.51%) | 10 (19.23%) |
| Final eTICI (categories 2c and 3) n (%) | 26 (61.9%) | 29 (69%) | 31 (56.3%) |
| Final eTICI (categories 2b and 3) n (%) | 39 (92.3%) | 38 (90.3%) | 41 (82%) |

TABLE 4
Primary and secondary outcome measures. IQR: inter quartile range.
In column Effects Placebo vs ApTOLL 0.2 mg/kg, positive % values
represent a decrease while negative % values represent an increase.

| | | | |
|---|---|---|---|
| ApTOLL | ApTOLL | | Effects Place- |
| 0.05 mg/kg | 0.2 mg/kg | Placebo | bo vs ApTOLL |
| (N = 42) | (N = 42) | (N = 55) | 0.2 mg/kg |

Primary outcome measures (along duration of clinical trial)

| | | | | |
|---|---|---|---|---|
| Death of any cause, n (%) | 11 (26.2%) | 2 (4.8%) | 10 (18.2%) | 73.63% |
| | | | | (18.2% to 4.8%) |
| Intracranial Hemorrhage, n | 20 (47.62%) | 17 (40.47%) | 26 (47.27%) | 14.39% |
| (%) | | | | (47.27% to 40.47%) |
| Symptomatic Intracranial | 3 (7.1%) | 2 (4.8%) | 4 (7.3%) | 34.25% |
| Hemorrhage, n (%) | | | | (7.3% to 4.8%) |
| Brain edema, n (%) | 2 (4.76%) | 1 (2.38%) | 4 (7.3%) | 67.40% |
| | | | | (7.3% to 2.38%) |
| Secondary outcome measures | | | | |
| Final Infarct Volume at 72 | 46.00 | 23.50 | 44.00 | 46.59% |
| hours (median [IQR]) (ml) | [18.00, 100.00] | [13.00, 42.00] | [26.00, 89.00] | (44.00 to 23.5) |
| NIHSS at 72 hours (median | 8.00 | 3.00 | 7.00 | 57.14% |
| [IQR]) | [3.00, 17.00] | [0.00, 11.00] | [3.00, 17.00] | (7.00 to 3.00) |
| mRS score 90 d (median | 3.00 | 2.00 | 3.00 | — |
| [IQR]) | [2.00, 5.00] | [1.00, 3.00] | [1.00, 4.00] | |
| 0 | 3 (7.50%) | 7 (16.67%) | 4 (7.55%) | −120.8% |
| | | | | (7.55 to 16.67) |
| 1 | 6 (15.00%) | 12 (28.57%) | 10 (18.87%) | −51.4% |
| | | | | (18.87 to 28.57) |
| 2 | 6 (15.00%) | 8 (19.05%) | 11 (20.75%) | 8.19% |
| | | | | (20.75 to 19.05) |
| 3 | 11 (27.50%) | 9 (21.43%) | 9 (16.98%) | −26.21% |
| | | | | (16.98 to 21.43) |
| 4 | 1 (2.50%) | 4 (9.52%) | 7 (13.21%) | 27.93% |
| | | | | (13.21 to 9.52) |
| 5 | 4 (10.00%) | | 3 (5.66%) | 78.97% |
| 6 | 9 (22.50%) | 2 (4.76%) | 9 (16.98%) | (22.64 to 4.76) |
| Missing | | | 1 | |
| mRS score 90 d of 0-2, n (%) | 15 (37.5%) | 27 (64.29%) | 25 (47.17%) | −36.29% |
| | | | | (47.17% to 64.29%) |
| mRS score 90 d of 0-1, n (%) | 9 (22.5%) | 19 (45.24%) | 14 (26.42%) | −71.23% |
| | | | | (26.42% to 45.24%) |
| mRS score 90 d of 3-6, n (%) | 25 (62.5%) | 15 (35.71%) | 28 (52.83%) | 32.40% |
| | | | | (52.83% to 35.71%) |

TABLE 5
Secondary outcome measures over time.

| | | ApTOLL | |
|---|---|---|---|
| | | 0.2 mg/kg | Placebo |
| | | (N = 42) | (N = 55) |
| Infarct Volume | Infarct Volume at baseline | 14 | 20.5 |
| | (median) (ml) | | |
| | Final Infarct Volume at | 23.5 | 44 |
| | 72 h (median) (ml) | | |
| | Effect baseline vs 72 h | −67.86% | −114.63% |
| | | (14 to 23.5) | (20.5 to 44) |
| | Effect placebo vs ApTOLL | 40.80% | |
| | 0.2 mg/kg | (−114.63 to −67.86) | |
| mRS score | Pre-stroke mRS score 0-2 | 42 (100%) | 53 (98.15%) |
| | (at baseline), n (%) | | |
| | mRS score 0-2 at 90 d, | 27 (64.29%) | 25 (47.17%) |
| | n (%) | | |
| | Effect baseline vs 90 d | 35.71% | 51.94% |
| | | (100% to 64.29%) | (98.15% to 47.17%) |

| | |
|---|---|
| Effect placebo vs ApTOLL | 31.25% |
| 0.2 mg/kg | (51.94% to 35.71%) |

### Example 2: Comparison of ApTOLL (APRIL Study) Vs. Nerinetide (ESCAPE-NA1 Study)

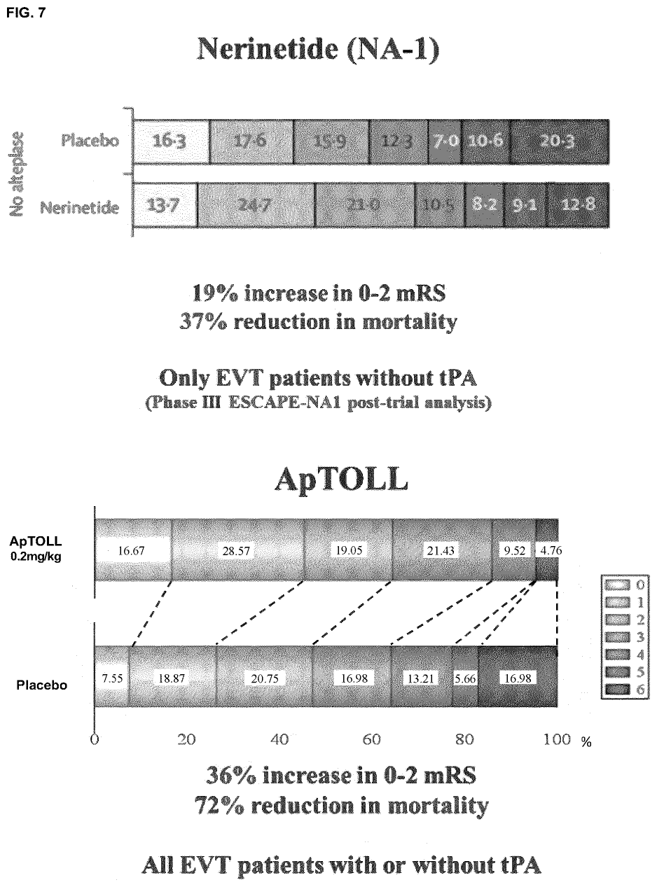

Nerinetide (NA-1) is a neuroprotectant focused on the inhibition of post-synaptic density protein 95 implicated in the excitotoxic process developed after stroke. The global results of ESCAPE-NA1 clinical trial with 1105 patients were negative since nerinetide did not improve the global proportion of patients achieving good clinical outcomes after EVT compared with patients receiving placebo. This negative effect could be explained by the fact that the population treated with EVT and EVT+tPA were mixed in the final analysis but, in a subsequent sub-analysis, investigators observed evidence of nerinetide effect modification resulting in inhibition of Nerinetide effect in patients receiving alteplase (Hill M et al., 2020). The results considering only patients not treated with tPA are summarized in FIG. 7, with a 19% of increase in 0-2 mRS and a 37% reduction of mortality.

It is noted that ESCAPE-NA1 was a phase III clinical trial with 1105 patients but with very limited results since effects were observed only in patients not treated with tPA. Therefore, the applicability of nerinetide to the clinical practice in the future would be limited to patients not eligible to tPA treatment. Contrarily, APRIL study conducted with only 151 patients resulted in a 36% increase in 0-2 mRS and a 72% reduction in mortality over patients treated or not treated with tPA.

### Example 3: Mutational Analysis of ApTOLL

The effects of percentage of sequence identity and extension at the 5′ end and 3′ end of ApTOLL sequence (SEQ ID NO: 1) were tested experimentally.

First, six variants of ApTOLL sequence presented in Table 6 were obtained and tested. The variants range in sequence identity to SEQ ID NO: 1 between 89.83% and 81.53% and include mutations with respect to the sequence of the aptamer of SEQ ID NO: 1 such as additions at the 5′ end of the sequence, additions both at the 5′ end and 3′ end of the sequence, internal deletions, conservative substitutions, and non-conservative substitutions.

TABLE 6
Mutants (Mut) of ApTOLL sequence.
The mutations with respect to ApTOLL sequence
are indicated by boldface.

| | | % identity to | Nucleotides |
|---|---|---|---|
| Aptamer | Sequence | SEQ ID NO: 1 | number |
| ApTOLL | GGTGTGCCAATAAACCATATCGCCGC | 100.00 | 59 |
| (SEQ ID NO: 1) | GTTAGCATGTACTCGGTGGCCCTAAA | | |
| | TACGAG | | |
| ApTOLL-Mut 1 | CCTGTGCCAATAAAGGATACCGCCGC | 91.53 | 59 |
| (SEQ ID NO: 17) | GTTAGCATGTACTCGGTTGGCCCTAA | | |
| | ATACGAG | | |
| ApTOLL-Mut 2 | GGTGTGCCAATAAACCATATCGCCGC | 91.53 | 59 |
| (SEQ ID NO: 18) | GTTAGCATGTACAGGGTTGGCCCCAA | | |
| | ATACCTG | | |
| ApTOLL-Mut 3 | TCCTGTGCCAATAAAGGATACCGCCG | 90.76 | 60 |
| (SEQ ID NO: 19) | CGTTAGCATGTACTCGGTTGGCCCTA | | |
| | AATACGAG | | |
| ApTOLL-Mut 4 | CCTGTGCCAATAAAGGATACCGCCGC | 90.60 | 58 |
| (SEQ ID NO: 20) | GTTAGCATGTACTCGGTTGGCCC-AA | | |
| | ATACGAG | | |
| ApTOLL-Mut 5 | TCCTGTGCCAATAAAGGATACCGCCG | 90.00 | 61 |
| (SEQ ID NO: 21) | CGTTAGCATGTACTCGGTTGGCCCTA | | |
| | AATACGAGT | | |
| ApTOLL-Mut 6 | GGTGTGCCAATAAACCATACCGCCGC | 89.83 | 59 |
| (SEQ ID NO: 22) | GTTAGCATGTACCTGGTTGGCCCCAA | | |
| | ATACAGG | | |

The antagonist activity against TLR4 was assessed with Secreted Embryonic Alkaline Phosphatase (SEAP) assay protocol in HEK-Blue hTLR4 cells (InvivoGen, Cat. code Hkb-htlr4). LPS-EK up (InvivoGen Cat. code tlrl-peklps) is the TLR4 agonist control. The HEK-Blue hTLR4 cells were obtained by co-transfection of the human TLR4, MD-2 and CD14 co-receptor genes, and an inducible SEAP reporter gene into HEK293 cells. The SEAP reporter gene was placed under the control of an IL-12 p40 minimal promoter fused to five NF-κB and AP-1-binding sites. Stimulation with a TLR4 ligand activates NF-κB and AP-1, which induces the production of SEAP. The seeded HEK-Blue hTLR4 cells in a P96 plate have their TLR4 receptor activated with the addition of LPS-EK up. One hour later, the aptamers were added in two final concentrations (20 nM, 200 nM) and the quantification of reporter protein SEAP expression was made after 16-20 hours.

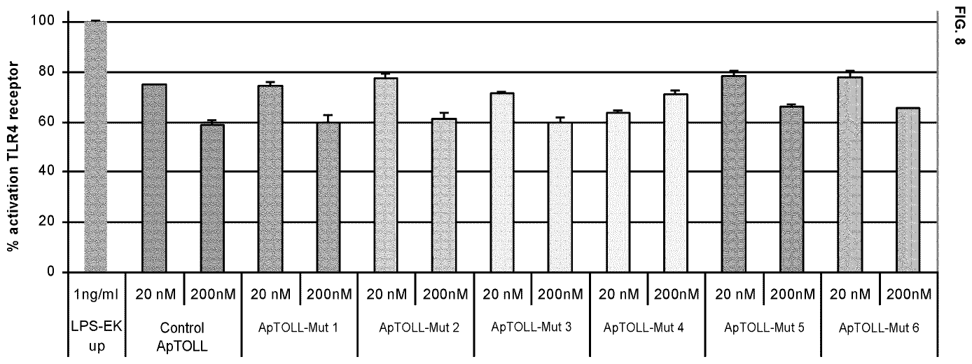

FIG. 8 shows the activity of SEAP reporter protein produced by the TLR4 receptor activation. The natural agonist ligand of TLR4 is LPS (bacterial lipopolysaccharide); in this assay LPS-EK up was used. The agonist control bar on the left represents that the TLR4 receptor is 100% activated with the mentioned agonist. When the aptamers (control ApTOLL and ApTOLL-Mut 1-6) are added to the activated cells, the TLR4 activity drops and the aptamers bars become shorter than the agonist control bar. Consequently, the aptamers have concentration dependent TLR4 antagonist activity, except for ApTOLL-Mut 4, which is not concentration dependent. The tested mutants have similar behavior, and therefore, FIG. 8 shows that all the ApTOLL variants (ApTOLL-Mut, SEQ ID NO: 17-22) tested had TLR4 antagonist activity comparable to that of ApTOLL aptamer (SEQ ID NO: 1).

Furthermore, a competition assay for TLR4 receptor for these aptamers was also assessed. ELONA-cell assay was made using ApTOLL aptamer (SEQ ID NO: 1) which was modified with a digoxigenin tag (ApTOLL-Dig) and HEK-Blue hTLR4 cells. Equimolecular mixes containing ApTOLL-Dig (100 nM) and each ApTOLL-Mut 1-6 (100 nM), respectively, were used to identify the possible reduction of digoxigenin related signal in respect to the positive ApTOLL-Dig control (100 nM). The positive ApTOLL-Dig control and each mix ApTOLL-Dig/ApTOLL-Mut were added into wells and incubated at 37° C. in CO₂ incubator for 15 minutes; then, the wells with cells were washed with PBS, anti-dig antibody was added, and finally ApTOLL-Dig was detected using ABTS protocol (Roche ref. 11684302001).

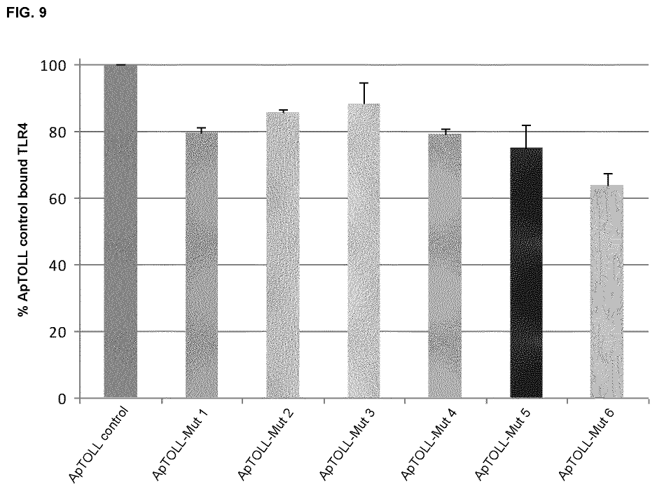

FIG. 9 depicts the results of the competition assay, which show that the binding percentage of ApTOLL control aptamer (SEQ ID NO: 1) to TLR4 receptor is 100% when it is added to the cell. Contrarily, when the ApTOLL-Mut aptamers (1-6) in equimolecular mix are respectively added to the cell, the binding percentage bar of ApTOLL aptamer is decreased, thus implying that the ApTOLL-Mut aptamers (1-6) are competing for the same TLR4 receptor binding site than the ApTOLL control aptamer. The ApTOLL-Mut 6 is the aptamer that obtained higher binding affinity to TLR4 receptor (35.9%), and ApTOLL-Mut 2 the lowest. ApTOLL-Mut 1 and ApTOLL-Mut 4 obtained a similar binding percentage (20% and 21%, respectively). Therefore, experimental data presented in FIG. 9 shows that the ApTOLL-Mut aptamers (SEQ ID NO: 17-22) competed with ApTOLL (SEQ ID NO: 1), which would indicate that they are structurally similar and therefore bind to the same binding site on TLR4 as the original ApTOLL aptamer.

Furthermore, the effect of lengthening the 5′ end and the 3′ end of ApTOLL (SEQ ID NO: 1) and simultaneously introducing mutations was also tested. Three variants of SEQ ID NO: 1 were tested (see Table 7, below):

Aptamer 4F (SEQ ID NO: 4): it has 13 extra nucleotides at the 5′ end and 4 extra nucleotides at the 3′ end compared to ApTOLL sequence (SEQ ID NO: 1). Sequence identity to SEQ ID NO: 1 would be 100% if only the central region was considered. Considering the entire sequence the percentage of sequence identity is 77.6%.

Aptamers 4F-Mut2 and 4F-Mut3 (SEQ ID NO: 23 and 24): these two aptamers would correspond to the most divergent sequences with respect to the ApTOLL sequence (SEQ ID NO: 1).

To test whether any 5′ end extension or 3′ end extension would work, the additional 5′ end and 3′ end regions of 4F-Mut2 and 4F-Mut3 extremely diverge from the extensions in aptamer 4F. The 5′ extensions in 4F-Mut2 and 4F-Mut3 have 0% sequence identity with respect to the 5′ end extension of aptamer 4F. Likewise, the 3′ extensions in 4F-Mut2 and 4F-Mut3 have 0% sequence identity with respect to the 3′ end extension of aptamer 4F.

| | 5′ Additional | 3′ Additional |
|---|---|---|
| | Sequence | Sequence |
| 4F | GCGGATGAAGACT | CAAC |
| 4F-Mut2 | TTTTTCTTTTTTC | TTTT |
| 4F-Mut3 | ATAAGCAGGAGTC | TGGT |

TABLE 7
The regions of 4F, 4F-Mut2 and 4F-Mut3 homologous to ApTOLL (SEQ ID NO: 1) are
shaded in gray, while the mutations in 4F-Mut2 and 4F-Mut3 with respect to ApTOLL (SEQ ID NO:
1) are indicated by boldface.

| | | Sequence | | |
|---|---|---|---|---|
| | | identity (%) | | |
| | | Partial | Extra | Extra |
| Aptamer | 5′-3′ sequence | (Full) | 5′ | 3′ |
| ApTOLL | GGTGTGCCAATAAACCATATCGCCGCG | | | |
| (SEQ ID NO: 1) | TTAGCATGTACTCGGTTGGCCCTAAAT | | | |
| | ACGAG | | | |
| 4F (SEQ ID NO: 4) | 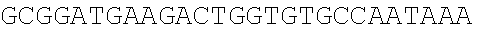 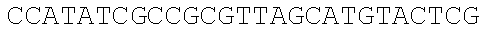 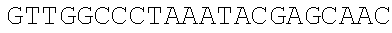 | 100% (77%) | 13 | 4 |
| 4F-Mut2 (SEQ ID NO: 23) | 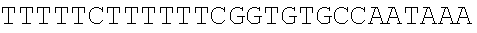 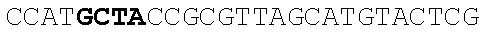 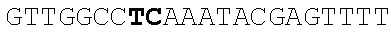 | 90% (69.7%) | 13 | 4 |
| 4F-Mut3 (SEQ ID NO: 24) | 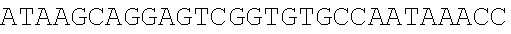 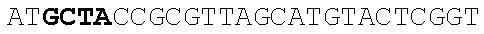 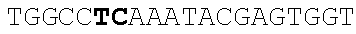 | 90% (69.7%) | 13 | 4 |

The ability of the above aptamers to inhibit TLR4 and the ability of the above aptamers to bind to TLR4 (antagonist activity against TLR4) were determined by means of assays disclosed above, and results are shown in Table 8.

TABLE 8
TLR4 antagonist activity of 4F, 4F-Mut2 and 4F-Mut3 aptamers.

| | | | TLR4 | Error | TLR4 antagonist |
|---|---|---|---|---|---|
| Sample | Concentration | | activity (%) | (%) | activity (%) |
| 4F-Mut2 | 20 | nM | 74.4 | 3.0 | Yes |
| | 200 | nM | 69.8 | 5.0 | |
| 4F-Mut3 | 20 | nM | 77.6 | 4.0 | Yes |
| | 200 | nM | 79.0 | 8.0 | |
| 4F-T | 20 | nM | 71.8 | 6.0 | Yes |
| (Control) | 200 | nM | 57.3 | 3.0 | |

Therefore, the addition of nucleotides at the 5′ end and 3′ end of SEQ ID NO: 1 does not affect formation of the effective binding structure and its activity, as confirmed experimentally in Table 8. The results from Table 8 confirm that both the 4F-Mut2 and the 4F-Mut3 aptamers, which have 90% sequence identity with SEQ ID NO: 1 and also have the addition of 13 nucleotides at the 5′ end and of 4 nucleotides at 3′ end the aptamer of SEQ ID NO: 1, maintain the capability of inhibiting TLR4 and the capability of binding to TLR4 (antagonist activity against TLR4), similar to their original aptamer of SEQ ID NO: 1.

Hence, a variant aptamer having at least 90% sequence identity with the original nucleic acid sequence (SEQ ID NO: 1) (or at least 69% sequence identity considering the whole sequence) and optionally extended at the 5′ and 3′ ends by 1-13 or 1-4 nucleotide sequences, respectively, maintains the functions (i.e., the capability of binding specifically to and inhibiting TLR4) of the original nucleic acid sequence.

### REFERENCES

WO2015197706A1 (AptaTargets SL)
WO2020230108A1 (AptaTargets SL)
WO2020230109A1 (AptaTargets SL)
Hernández-Jiménez M, et al. First-in-human phase I clinical trial of a TLR4-binding DNA aptamer, ApTOLL: Safety and pharmacokinetics in healthy volunteers. Mol Ther Nucleic Acids. 2022; 28:124-135. doi:10.1016/j.omtn.2022.03.005
García-Culebras A, et al. Toll-Like Receptor 4 Mediates Hemorrhagic Transformation After Delayed Tissue Plasminogen Activator Administration in In Situ Thromboembolic Stroke. Stroke. 2017 June; 48 (6): 1695-1699. doi:10.1161/STROKEAHA.116.015956
Berge E, et al. European Stroke Organisation (ESO) guidelines on intravenous thrombolysis for acute ischaemic stroke. European Stroke Journal. 2021; 6 (1): I-LXII. doi:10.1177/2396987321989865
Jovin T G, et al. Thrombectomy within 8 Hours after Symptom Onset in Ischemic Stroke. N Engl J Med 2015; 372:2296-2306. doi:10.1056/NEJMoa1503780
Lyden P, et al. Improved reliability of the NIH Stroke Scale using video training. NINDS TPA Stroke Study Group. Stroke. 1994; 25 (11): 2220-2226. doi:10.1161/01.str.25.11.2220
Van Swieten J C, et al. Interobserver agreement for the assessment of handicap in stroke patients. Stroke. 1988; 19 (5): 604-607. doi:10.1161/01.str.19.5.604
Chen S, et al. Cerebral edema formation after stroke: emphasis on blood-brain barrier and the lymphatic drainage system of the brain. Front. Cell. Neurosci. 15:716825. doi: 10.3389/fncel.2021.716825
Pexman J H, et al. Use of the Alberta Stroke Program Early CT Score (ASPECTS) for assessing CT scans in patients with acute stroke. AJNR Am J Neuroradiol. 2001; 22 (8): 1534-1542
Olivé-Gadea M, et al. Defining a target population to effectively test a neuroprotective drug. Stroke. 2021; 52 (2): 505-510. doi:10.1161/STROKEAHA.120.032025
Turc G, et al. European Stroke Organisation (ESO)-European Society for Minimally Invasive Neurological Therapy (ESMINT) guidelines on mechanical thrombectomy in acute ischemic stroke. J Neurointerv Surg. 2019; 11 (6): 535-538. doi:10.1136/neurintsurg-2018-014568
Hill M et al. Efficacy and safety of nerinetide for the treatment of acute ischaemic stroke (ESCAPE-NA1): a multicentre, double-blind, randomised controlled trial. 2020. The Lancet. 395 (10227): 878-887. doi:10.1016/S0140-6736 (20) 30258-0

1. An aptamer with SEQ ID NO: 1 for use in reducing the risk of intracranial hemorrhages after an acute ischemic stroke in a subject, wherein the aptamer is administered after the acute ischemic stroke at a dose of at least about 0.2 mg/kg, and wherein the administration of the aptamer reduces the risk of intracranial hemorrhages in at least about a 14% with respect to a subject not treated with the aptamer.

2. The aptamer for use according to claim 1, wherein the intracranial hemorrhages are symptomatic intracranial hemorrhages, and wherein the administration of the aptamer reduces the risk of symptomatic intracranial hemorrhages in at least about a 34% with respect to a subject not treated with the aptamer.

3. An aptamer with SEQ ID NO: 1 for use in improving the neurological recovery after an acute ischemic stroke in a subject, wherein the improvement of neurological recovery is mediated by a reduction of the risk of intracranial hemorrhages, wherein the aptamer is administered after the acute ischemic stroke at a dose of at least about 0.2 mg/kg, and wherein the aptamer:
(i) reduces the NIHSS score, and
(ii) reduces the mRS score to 0-2,
with respect to a subject not treated with the aptamer.

4. The aptamer for use according to claim 3, wherein the reduction in the NIHSS score at 72 hours is of at least about 57% with respect to a subject not treated with the aptamer.

5. The aptamer for use according to any of claims 3-4, wherein the reduction in the mRS score to 0-2 at 90 days is of at least about 36% with respect to a subject not treated with the aptamer.

6. The aptamer for use according to any of claims 1-5, wherein the aptamer reduces brain edema in at least about a 67% with respect to a subject not treated with the aptamer.

7. The aptamer for use according to any of claims 1-6, wherein the aptamer is administered within about 8 hours from stroke onset, particularly within about 4 hours from stroke onset.

8. The aptamer for use according to any of claims 1-7, wherein the aptamer is administered in combination with artery recanalization.

9. The aptamer for use according to claim 8, wherein the aptamer is administered in combination with endovascular treatment.

10. The aptamer for use according to claim 9, wherein the aptamer is administered prior or concurrently with the endovascular treatment.

11. The aptamer for use according to claim 10, wherein the aptamer is administered less than about 4 hours prior to endovascular treatment.

12. The aptamer for use according to any of claims 8-11, wherein the aptamer is administered in combination with pharmacological thrombolysis, particularly the administration of tissue plasminogen activator.

13. The aptamer for use according to claim 12, wherein the aptamer is administered concurrently or after the administration of pharmacological thrombolysis.

14. The aptamer for use according to any of claims 1-13, wherein the aptamer is administered intravenously by infusion for about 30 minutes.

15. The aptamer for use according to any of claims 1-14, wherein the subject:
i) is between about 18 and about 90 years of age;
ii) has a baseline NIHSS of between about 8 points and about 25 points;
iii) has a pre-stroke mRS score of between 0 points and about 2 points;
iv) has an infarct volume of between about 5 cc and about 70 cc;
v) has an occlusion in the terminal internal carotid artery, M1 or M2 segments of the middle cerebral artery;
vi) has a mTICI score of 0 or 1;
vii) has a volume of diffusion-weighted imaging restriction between about 5 mL and about 70 mL;
viii) has an ASPECTS between about 6 and about 10; or
ix) any combination thereof,
at stroke onset.
